(19) United States
(12) Patent Application Publication
(10) Pub. No.: US 2026/0125462 A1
(43) Pub. Date: May 7, 2026

(54) COMPOUNDS AND METHODS TARGETING INTERLEUKIN-19

(71) Applicant: Eli Lilly and Company, Indianapolis, IN (US)

(72) Inventors: Richard Earl HIGGS, JR., Zionsville, IN (US); Robert John KONRAD, Carmel, IN (US); Brian Jeffrey NICKOLOFF, Carmel, IN (US); Robert William SIEGEL, II, Fountaintown, IN (US)

(21) Appl. No.: 19/323,384

(22) Filed: Sep. 9, 2025

Related U.S. Application Data
(62) Division of application No. 18/159,393, filed on Jan. 25, 2023, now Pat. No. 12,459,997.
Application No. 18/159,393 is a continuation of application No. 16/903,731, filed on Jun. 17, 2020, now Pat. No. 11,591,387.
Application No. 16/903,731 is a continuation of application No. PCT/US2019/013565, filed on Jan. 15, 2019.
(60) Provisional application No. 62/618,200, filed on Jan. 17, 2018.

(51) Int. Cl. C07K 16/24

(52) U.S. Cl. CPC C07K 16/244; C07K 2317/34; C07K 2317/565

(57) ABSTRACT

The present invention provides compounds and methods targeting human interleukin-19, including therapeutic antibodies, pharmaceutical compositions and diagnostic applications useful in the field of immune-mediated diseases including psoriasis, atopic dermatitis, psoriatic arthritis, bronchial asthma and diabetic nephropathy.

The present application is filed along with a Sequence Listing in ST.26 XML format. The Sequence Listing is provided as a file titled “X20963A.xml” created Jan. 6, 2023 and is 397,757 bytes in size. The Sequence Listing information in ST.26 XML format is incorporated by reference in its entirety.

The present invention is in the field of medicine. More particularly, the present invention relates to compounds, pharmaceutical compositions, and methods which include an antibody directed against human interleukin-19 (IL-19). The compounds and methods of the present invention are expected to be useful in the field of autoimmune and chronic inflammatory diseases (collectively referred to herein as, immune-mediated diseases), particularly diseases such as psoriasis (Ps0), atopic dermatitis (AD), diabetic nephropathy (DN), bronchial asthma (BA), psoriatic arthritis (PsA) and the like, including treatment thereof and diagnostic applications relating thereto.

Interleukin-19 (IL-19) is a cytokine reported to belong to the interleukin-10 cytokine family (which includes IL-10, 20, 22 and 26 as well as some virus-encoded cytokines). IL-19 has been reported to have involvement in the IL-20R complex signaling pathway and to be expressed in resting monocytes, macrophages, B cells, and epithelial cells including keratinocytes.

Autoimmune diseases arise from the body's production of an immune response against its own tissue. Autoimmune diseases are often chronic and can be debilitating and even life-threatening. Ps0 is a chronic autoimmune disease with systemic manifestations including psoriatic arthritis, cardiovascular disease, metabolic syndrome and affective disorders. AD, along with many other forms of chronic autoimmune diseases such as Ps0, RA, AxSpA and PsA, affect the axial and/or peripheral skeleton.

Current FDA approved treatments for immune-mediated diseases include corticosteroids, often used to treat acute inflammation, and bioproducts targeting TNFα or interleukin-12 and 23. Although these treatments have demonstrated efficacy in reducing symptoms for a subset of patients, a large percentage of patients remain nonresponsive or experience a loss of response to the currently available treatments. For autoimmune diseases such as Ps0, ixekizumab is an FDA approved therapeutic antibody targeting IL-17A in which 90% of patients achieved a 75% reduction in the Psoriasis Assessment Skin Involvement (PASI) score (e.g. PASI 75). However, PASI assessments rely on subject inputs that can be difficult to assess in certain circumstances. To date, an objective, sensitive, and reproducible blood-based biomarker for assessing and informing clinical management of Ps0, and other immune-mediated diseases such as AD, DN and BA does not exist. Thus, there remains an unmet need for compounds, pharmaceutical compositions, and methods useful as therapeutics for, and/or in diagnostic applications relating to, immune-mediated diseases such as Ps0, AD, BA, DN and the like.

Accordingly, in certain embodiments, the present invention provides antibodies directed against human IL-19. According to some embodiments, the present invention provides antibodies which comprise a light chain variable region (LCVR) and a heavy chain variable region (HCVR), wherein the LCVR comprises complementarity determining regions (CDRs) LCDR1, LCDR2 and LCDR3 and the HCVR comprises CDRs HCDR1, HCDR2 and HCDR3 selected from the groupings of CDR combinations provided in Table 1, 2 or 3. In some embodiments, the LCVR comprises CDRs LCDR1, LCDR2 and LCDR3 and the HCVR comprises CDRs HCDR1, HCDR2 and HCDR3 having amino acid sequences with at least 95% homology to the amino acid sequences selected from the groupings of CDR combinations provided in Table 1, 2 or 3. According to particular embodiments, the present invention also provides antibodies comprising a LCVR and a HCVR selected from:
a. the LCVR having the amino acid sequence of SEQ ID NO: 66 and the HCVR having the amino acid sequence of SEQ ID NO: 70;
b. the LCVR having the amino acid sequence of SEQ ID NO: 74 and the HCVR having the amino acid sequence of SEQ ID NO: 78;
c. the LCVR having the amino acid sequence of SEQ ID NO: 82 and the HCVR having the amino acid sequence of SEQ ID NO: 86;
d. the LCVR having the amino acid sequence of SEQ ID NO: 34 and the HCVR having the amino acid sequence of SEQ ID NO: 38;
e. the LCVR having the amino acid sequence of SEQ ID NO: 42 and the HCVR having the amino acid sequence of SEQ ID NO: 46;
f. the LCVR having the amino acid sequence of SEQ ID NO: 50 and the HCVR having the amino acid sequence of SEQ ID NO: 54; and
g. the LCVR having the amino acid sequence of SEQ ID NO: 58 and the HCVR having the amino acid sequence of SEQ ID NO: 62.

According to even more particular embodiments, the present invention also provides antibodies comprising a LC and a HC selected from, or having amino acid sequences with at least 95% homology to the amino acid sequences of:
a. the LC having the amino acid sequence of SEQ ID NO: 2 and the HC having the amino acid sequence of SEQ ID NO: 6;
b. the LC having the amino acid sequence of SEQ ID NO: 10 and the HC having the amino acid sequence of SEQ ID NO: 14;
c. the LC having the amino acid sequence of SEQ ID NO: 18 and the HC having the amino acid sequence of SEQ ID NO: 22;
d. the LC having the amino acid sequence of SEQ ID NO: 26 and the HC having the amino acid sequence of SEQ ID NO: 30;
e. the LC having the amino acid sequence of SEQ ID NO: 310 and the HC having the amino acid sequence of SEQ ID NO: 311; and
f. the LC having the amino acid sequence of SEQ ID NO: 312 and the HC having the amino acid sequence of SEQ ID NO: 313.

According to particular embodiments, the present invention provides human IL-19 neutralizing antibodies having a LCVR and a HCVR, wherein the LCVR comprises CDRs (LCDR1, LCDR2 and LCDR3) and the HCVR comprises CDRs (HCDR1, HCDR2 and HCDR3) selected from, or having amino acid sequences with at least 95% homology to the amino acid sequences of, the CDR combinations provided in Table 1, 2 or 3. In particular embodiments, the human IL-19 neutralizing antibodies of the present invention comprise a LCVR and a HCVR selected from:
a. the LCVR having the amino acid sequence of SEQ ID NO: 66 and the HCVR having the amino acid sequence of SEQ ID NO: 70;
b. the LCVR having the amino acid sequence of SEQ ID NO: 74 and the HCVR having the amino acid sequence of SEQ ID NO: 78;
c. the LCVR having the amino acid sequence of SEQ ID NO: 82 and the HCVR having the amino acid sequence of SEQ ID NO: 86;
d. the LCVR having the amino acid sequence of SEQ ID NO: 34 and the HCVR having the amino acid sequence of SEQ ID NO: 38;
e. the LCVR having the amino acid sequence of SEQ ID NO: 42 and the HCVR having the amino acid sequence of SEQ ID NO: 46;
f. the LCVR having the amino acid sequence of SEQ ID NO: 50 and the HCVR having the amino acid sequence of SEQ ID NO: 54; and
g. the LCVR having the amino acid sequence of SEQ ID NO: 58 and the HCVR having the amino acid sequence of SEQ ID NO: 62.

According to even more particular embodiments, the present invention also provides human IL-19 neutralizing antibodies having a LC and a HC selected from, or having amino acid sequences with at least 95% homology to the amino acid sequences of:
a. the LC having the amino acid sequence of SEQ ID NO: 2 and the HC having the amino acid sequence of SEQ ID NO: 6;
b. the LC having the amino acid sequence of SEQ ID NO: 10 and the HC having the amino acid sequence of SEQ ID NO: 14;
c. the LC having the amino acid sequence of SEQ ID NO: 18 and the HC having the amino acid sequence of SEQ ID NO: 22;
d. the LC having the amino acid sequence of SEQ ID NO: 26 and the HC having the amino acid sequence of SEQ ID NO: 30; and
e. the LC having the amino acid sequence of SEQ ID NO: 310 and the HC having the amino acid sequence of SEQ ID NO: 311; and
f. the LC having the amino acid sequence of SEQ ID NO: 312 and the HC having the amino acid sequence of SEQ ID NO: 313.

In embodiments, antibodies of the present invention comprise an IgG1 heavy chain. According to some embodiments, the antibodies further comprise kappa light chains.

According to some aspects of the present invention, human IL-19 antibodies, including human IL-19 neutralizing antibodies, are provided which bind human IL-19 within an epitope region comprising at least one or more of amino acid residues: 95-102; 67-75; 125-136; 67-75 and 125-136; 90-100; 42-60; 90-107; 149-160; 42-60, 90-107 and 149-160 of human IL-19 as given by SEQ ID NO. 1 (as determined my methods set forth in the present disclosure). In an embodiment, the present invention provides IL-19 antibodies that bind human IL-19 within an epitope region of human IL-19 which bins with an antibody provided herein.

According to some embodiments, the IL-19 antibodies of the present invention are useful in the treatment of immune-mediated diseases. In some more specific embodiments, the immune-mediated diseases are at least one of Ps0, AD, PSA, BA and/or DN. According to other embodiments of the present invention, the IL-19 antibodies of the present invention are useful in diagnostic applications for autoimmune diseases. In some more specific embodiments, the immune-mediated diseases are at least one of Ps0, AD and/or DN.

The present invention further provides pharmaceutical compositions comprising an IL-19 antibody of the present invention and one or more pharmaceutically acceptable carriers, diluents or excipients. Further, the present invention provides a method of treating an immune-mediated disease, such as Ps0, AD and/or DN, comprising administering to a patient in need thereof a pharmaceutical composition of the present invention.

In addition, the present invention provides a method of treating immune-mediated diseases. More particularly, the present invention provides a method of treating immune-mediated diseases, including Ps0, AD, PSA, BA or DN comprising administering to a patient in need thereof an effective amount of an IL-19 antibody of the present invention.

The present invention also provides an IL-19 antibody of the present invention for use in therapy. More particularly, the present invention provides an IL-19 antibody of the present invention for use in treatment of immune-mediated diseases including Ps0, AD, PsA and DN. In an embodiment, the present invention provides the use of an IL-19 antibody of the present invention in the manufacture of a medicament for the treatment of one or more immune-mediated diseases including Ps0, AD, PSA, BA and DN.

According to some embodiments, the present invention provides a method of detecting IL-19 in a patient sample comprising the steps of contacting the patient sample with a first antibody which binds a first epitope region of IL-19; contacting the patient sample with a second antibody which binds a second epitope region of IL-19 and has a detectable label; and detecting a signal provided by said detectable label. In some embodiments, the patient sample is one of blood, serum or plasma. According to some more specific embodiments, the first epitope region of IL-19 partially overlaps with the second epitope region of IL-19. Further, in some embodiments, said steps of contacting with the first and second antibodies occurs simultaneously. In some specific embodiments, the first antibody comprises a combination of LC and HC CDRs provided in Tables 1, 2 or 3. In some embodiments, the first antibody comprises a combination of LC and HC CDRs having 95% homology to the LC and HC CDRs provided in Tables 1, 2 or 3. In some specific embodiments, the second antibody comprises a combination of LC and HC CDRs provided in Tables 1, 2 or 3. In some embodiments, the second antibody comprises a combination of LC and HC CDRs having 95% homology to the LC and HC CDRs provided in Tables 1, 2 or 3. In more particular embodiments, the first and second antibodies do not bin together.

According to some embodiments of the present invention, a method of quantifying IL-19 in a patient sample is provided. Such method includes the steps of contacting the patient sample with a first antibody which binds a first epitope region of IL-19: contacting the patient sample with a second antibody which binds a second epitope region of IL-19 and said has a detectable label; and detecting the signal provided by said detectable label; contacting a control standard with a first antibody which binds the same first epitope region of IL-19 (as used in contacting the patient sample): contacting the control standard with a second antibody which binds the same second epitope region of IL-19 (as used in contacting the patient sample) and having a detectable label; and detecting a signal provided by said detectable signal. In some embodiments, the patient sample is one of blood, serum or plasma. According to some more specific embodiments, the first epitope region of IL-19 partially overlaps with the second epitope region of IL-19. Further, in some embodiments, said steps of contacting with the first and second antibodies occurs simultaneously. In some specific embodiments, the first antibody comprises a combination of LC and HC CDRs provided in Tables 1, 2 or 3. In some embodiments, the first antibody comprises a combination of LC and HC CDRs having 95% homology to the LC and HC CDRs provided in Tables 1, 2 or 3. In some specific embodiments, the second antibody comprises a combination of LC and HC CDRs provided in Tables 1, 2 or 3. In some embodiments, the second antibody comprises a combination of LC and HC CDRs having 95% homology to the LC and HC CDRs provided in Tables 1, 2 or 3. In more particular embodiments, the first and second antibodies do not bin together.

According to some embodiments, a method of diagnosing an immune-mediated disease is provided. Such method comprises the steps of contacting a patient sample with an IL-19 antibody and detecting binding between IL-19 in the patient sample and the antibody. According to some specific embodiments, the method of diagnosing includes diagnosing the patient as having; at risk for; in need of treatment for; and/or at risk of symptoms relating to an immune-mediated disease when the presence of IL-19 in the patient sample is detected as above a reference value. According to some more specific embodiments, such methods further include the steps of determining the reference value including the steps of contacting a control standard with a first antibody which binds the same first epitope region of IL-19 as used in contacting the patient sample: contacting the control standard with a second antibody having a detectable label and which binds the same second epitope region of IL-19 as used in contacting the patient sample; and detecting a signal provided by the detectable signal. In some embodiments, the first antibody comprises a combination of LC and HC CDRs provided in Tables 1, 2 or 3. In some embodiments, the first antibody comprises a combination of LC and HC CDRs having 95% homology to the LC and HC CDRs provided in Tables 1, 2 or 3. Some embodiments of the method of diagnosing an immune-mediated disease, provided herein, further includes the steps of contacting the patient sample with a second IL-19 antibody which binds a second epitope region of IL-19 and has a detectable label; and detecting a signal provided by the detectable label. In some specific embodiments, the IL-19 antibody comprises a combination of LC and HC CDRs provided in Table 1, 2 and 3. In some embodiments, the second IL-19 antibody comprises a combination of LC and HC CDRs provided in Table 1, 2 and 3. In some embodiments, the second antibody comprises a combination of LC and HC CDRs having 95% homology to the LC and HC CDRs provided in Table 1, 2, and 3. According to specific embodiments, the first epitope region of IL-19 partially overlaps with the second epitope region of IL-19. According to particular embodiments, the first and second antibodies do not bin together. According to further embodiments, the reference value is approximately 21 pg/mL. In further embodiments, the immune-mediated disease is one of Ps0, AD, PSA, BA and DN.

In even further embodiments, the present invention provides a method of treating an immune-mediated disease in a patient. Such methods comprise the steps of contacting a patient sample with an IL-19 antibody and detecting binding between IL-19 in the patient sample and the antibody; and diagnosing the patient as having; at risk for; in need of treatment for; and/or at risk of symptoms relating to an immune-mediated disease when the presence of IL-19 in the patient sample is detected as above a reference value. According to some more specific embodiments of the methods of treating provided herein, such methods further include the steps of determining the reference value including the further steps of contacting a control standard with a first antibody which binds the same first epitope region of IL-19 as used in contacting the patient sample; contacting the control standard with a second antibody having a detectable label and which binds the same second epitope region of IL-19 as used in contacting the patient sample; and detecting a signal provided by the detectable signal. In some specific embodiments, the IL-19 antibody comprises a combination of LC and HC CDRs provided in Tables 1, 2 and 3. In some embodiments, the IL-19 antibody comprises a combination of LC and HC CDRs having 95% homology to the LC and HC CDRs provided in Tables 1, 2 or 3. According to some embodiments, the reference value is approximately 21 pg/mL. In embodiments, the immune-mediated disease is one of Ps0, AD and DN. In some embodiments, the patient sample is one of blood, serum or plasma. According to some embodiments, the method further includes the steps of contacting the patient sample with a second IL-19 antibody which binds a second epitope region of IL-19 and has a detectable label and detecting a signal provided by the detectable signal. In even further embodiments, the second antibody comprises a combination of LC and HC CDRs provided in Tables 1, 2 or 3. In some embodiments, the second antibody comprises a combination of LC and HC CDRs having 95% homology to the LC and HC CDRs provided in Tables 1, 2 or 3. According to particular embodiments, the first and second antibodies do not bin together.

As used herein, an “antibody” is an immunoglobulin molecule comprising 2 HCs and 2 LCs interconnected by disulfide bonds. The amino terminal portion of each LC and HC includes a variable region of about 100-120 amino acids primarily responsible for antigen recognition via the CDRs contained therein. The CDRs are interspersed with regions that are more conserved, termed framework regions (“FR”). Each LCVR and HCVR is composed of 3 CDRs and 4 FRs, arranged from amino-terminus to carboxy-terminus in the following order: FR1, CDR1, FR2, CDR2, FR3, CDR3, FR4. The 3 CDRs of the LC are referred to as “LCDR1, LCDR2, and LCDR3,” and the 3 CDRs of the HC are referred to as “HCDR1, HCDR2, and HCDR3.” The CDRs contain most of the residues which form specific interactions with the antigen. The functional ability of an antibody to bind a particular antigen is largely influenced by the six CDRs. Assignment of amino acids to CDR domains within the LCVR and HCVR regions of the antibodies of the present invention is based on the well-known Kabat numbering convention (Kabat, et al., Ann. NY Acad. Sci. 190:382-93 (1971); Kabat et al., Sequences of Proteins of Immunological Interest, Fifth Edition, U.S. Department of Health and Human Services, NIH Publication No. 91-3242 (1991)), and North numbering convention (North et al., A New Clustering of Antibody CDR Loop Conformations, Journal of Molecular Biology, 406:228-256 (2011)).

LCs are classified as kappa or lambda, which are each characterized by a particular constant region as known in the art. HCs are classified as gamma, mu, alpha, delta, or epsilon, and define the isotype of an antibody as IgG, IgM, IgA, IgD, or IgE, respectively. The antibodies of the present invention include IgG HCs which can be further divided into subclasses, e.g., IgG1, IgG2, IgG3, IgG4. The carboxy-terminal portion of each HC defines a constant region primarily responsible for effector function. Particular embodiments of antibodies of the present invention may include one or more modifications in the constant region of each HC, for example that enhance or reduce effector function, as are known in the art.

The antibodies of the present invention are monoclonal antibodies. Monoclonal antibodies are antibodies derived from a single copy or clone including, for example, any eukaryotic, prokaryotic or phage clone, and not the method by which it is produced. Monoclonal antibodies can be produced, for example, by hybridoma technologies, recombinant technologies, phage display technologies, synthetic technologies, e.g., CDR-grafting, or combinations of such or other technologies known in the art.

Methods of producing and purifying antibodies are well known in the art and can be found, for example, in Harlow and Lane (1988), Antibodies. A Laboratory Manual, Cold Spring Harbor Laboratory Press, Cold Spring harbor, N.Y., chapters 5-8 and 15, ISBN 0-87969-314-2. For example, mice or rabbits may be immunized with human IL-19 and the resulting antibodies can be recovered, purified, and the amino acid sequences determined using conventional methods well known in the art. Likewise, a phage library may be screened, whereby thousands of Fab fragments are screened for interaction with human IL-19 and resulting interactions can be recovered, purified, and the amino acid sequences determined using conventional methods well known in the art, whereby initial lead antibodies can be constructed. According to possible embodiments, antibodies of the present invention may be engineered to contain one or more human framework regions surrounding CDRs derived from the non-human antibody. Human framework germline sequences can be obtained, for example, from ImMunoGeneTics (INGT) via their website, http://imgt.cines.fr, or from The Immunoglobulin FactsBook by Marie-Paule Lefranc and Gerard Lefranc, Academic Press, 2001, ISBN 012441351.

In particular embodiments of the present invention, the antibody, or the nucleic acid encoding same, is provided in isolated form. As used herein, the term “isolated” refers to a protein, peptide, or nucleic acid which is free or substantially free from other macromolecular species found in a cellular environment.

The antibodies of the present invention can be used in the treatment of patients. More particularly the antibodies of the present invention are expected to treat immune-mediated diseases or disorders, which include Ps0, AD, PSA, BA and DN. Although antibodies of the present invention are expected to be useful in the treatment of Ps0, AD and DN, such antibodies may also be useful in the treatment of other immune-mediated diseases, including RA, AxSpA and PsA and/or immune-mediated diseases specifically including epithelial cell involvement. As used interchangeably herein, “treatment” and/or “treating” and/or “treat” are intended to refer to all processes wherein there may be a slowing, interrupting, arresting, controlling, stopping, or reversing of the progression of the disorders described herein, but does not necessarily indicate a total elimination of all disorder symptoms. Treatment includes administration of an antibody of the present invention for treatment of a disease or condition in a human that would benefit from a reduction in IL-19 activity, and includes: (a) inhibiting further progression of the disease, i.e., arresting its development; and (b) relieving the disease, i.e., causing regression of the disease or disorder or alleviating symptoms or complications thereof.

As used interchangeably herein, the term “patient,” “subject,” and “individual,” refers to a human. In certain embodiments, the patient is further characterized with a disease, disorder, or condition (e.g., an autoimmune disorder) that would benefit from a reduction in IL-19 activity. In other embodiments, the patient is further characterized as being at risk of developing an immune-mediated disease, disorder, or condition that would benefit from a reduction in IL-19 activity.

A patient “sample” as used herein refers to a human sample. Non-limiting sources of a sample for use in the present invention include blood, plasma, serum, spinal fluid, lymph fluid, biopsy aspirates, ascites, fluidic extracts, solid tissue, the external sections of the skin, respiratory, intestinal, and genitourinary tracts, tears, saliva, milk, tumors, organs, cell cultures and/or cell culture constituents.

As used herein, the term “bind (or binds)” IL-19 refers to an interaction of an antibody with a epitope region of human IL-19. The term “epitope region” refers to specific amino acids comprising IL-19 which provide an antigenic determinant capable of specific binding to an IL-19 antibody. The amino acids of an epitope region provide chemically active surface groupings of IL-19 and form a specific three dimensional structure of IL-19, and may provide specific charge characteristics Binding may comprise interacting with the epitope region either through “conformational” or “linear” epitope binding of the antibody with human IL-19. Presented herein are exemplified embodiments of IL-19 antibodies that bind linear epitopes of human IL-19, and other exemplified embodiments of IL-19 antibodies that bind conformational epitopes. Conformational and nonconformational/linear epitopes may be distinguished in that the binding to the conformational epitope regions is lost in the presence of denaturing solvents whereas linear epitope regions is not. In a particular embodiment, the term “bind (or binds)” human IL-19 refers to an interaction with an epitope region comprising amino acid residues 95-102 of human IL-19, as determined my methods set forth in the present disclosure (residue numbering based on the exemplified human IL-19 of SEQ ID NO.1).

In a further embodiment, the term “bind (or binds)” human IL-19 refers to an interaction with an epitope region comprising amino acid residues 90-100 of human IL-19, as determined my methods set forth in the present disclosure (residue numbering based on the exemplified human IL-19 of SEQ ID NO.1). In another particular embodiment, the term “bind (or binds)” human IL-19 refers to an interaction with an epitope region comprising amino acid residues 67-75 of human IL-19, as determined my methods set forth in the present disclosure (residue numbering based on the exemplified human IL-19 of SEQ ID NO.1). In another embodiment, the term “bind (or binds)” human IL-19 refers to an interaction with an epitope region comprising amino acid residues 125-136 of human IL-19, as determined my methods set forth in the present disclosure (residue numbering based on the exemplified human IL-19 of SEQ ID NO.1). In another particular embodiment, the term “bind (or binds)” human IL-19 refers to an interaction with an epitope region comprising amino acid residues 67-75 and 125-136 of human IL-19, as determined my methods set forth in the present disclosure (residue numbering based on the exemplified human IL-19 of SEQ ID NO.1). In a further embodiment, the term “bind (or binds)” human IL-19 refers to an interaction with an epitope region comprising amino acid residues 42-60 of human IL-19, as determined my methods set forth in the present disclosure (residue numbering based on the exemplified human IL-19 of SEQ ID NO.1). In an embodiment, the term “bind (or binds)” human IL-19 refers to an interaction with an epitope region comprising amino acid residues 90-107 of human IL-19, as determined my methods set forth in the present disclosure (residue numbering based on the exemplified human IL-19 of SEQ ID NO.1). In an embodiment, the term “bind (or binds)” human IL-19 refers to an interaction with an epitope region comprising amino acid residues 149-160 of human IL-19, as determined my methods set forth in the present disclosure (residue numbering based on the exemplified human IL-19 of SEQ ID NO.1). In another particular embodiment, the term “bind (or binds)” human IL-19 refers to an interaction with an epitope region comprising amino acid residues 42-60, 90-107 and 149-160 of human IL-19, as determined my methods set forth in the present disclosure (residue numbering based on the exemplified human IL-19 of SEQ ID NO.1). It should be understood that there are known variations of human IL-19, for example resulting from splice variants. It is also understood that such known variants may result in altered residue numbering for residues described here (for example, as in relation to the residue numbering presented in SEQ ID NO.1). Although the residue numbering may be altered in some variants, the amino acids comprising the epitope region remain the same. The term “epitope region” as used herein refers to discrete, three-dimensional sites of an antigen that are recognized, either in total or in part, by the antibodies of the present invention.

An antibody of the present invention can be incorporated into a pharmaceutical composition which can be prepared by methods well known in the art and comprise an antibody of the present invention and one or more pharmaceutically acceptable carrier(s) and/or diluent(s) (e.g., Remington, The Science and Practice of Pharmacy, 22^{nd} Edition, Loyd V., Ed., Pharmaceutical Press, 2012, which provides a compendium of formulation techniques as are generally known to practitioners). Suitable carriers for pharmaceutical compositions include any material which, when combined with an antibody of the present invention, retains the molecule's activity and is non-reactive with the patient's immune system. A pharmaceutical composition comprising an antibody of the present invention can be administered to a patient at risk for, or exhibiting, diseases or disorders as described herein by parental routes (e.g., subcutaneous, intravenous, intraperitoneal, intramuscular, or transdermal). A pharmaceutical composition of the present invention contains an “effective” or “therapeutically effective” amount, as used interchangeably herein, of an antibody of the present invention. An effective amount refers to an amount necessary (at dosages and for periods of time and for the means of administration) to achieve the desired therapeutic result. An effective amount of an antibody may vary according to factors such as the disease state, age, sex, and weight of the individual, and the ability of the antibody to elicit a desired response in the individual. An effective amount is also one in which any toxic or detrimental effects of the antibody of the present invention are outweighed by the therapeutically beneficial effects.

The term percent homology, as used in the present disclosure, in the context of two or more amino acid sequence refers to two or more sequences having a specified percentage of amino acid residues that are the same, when compared and aligned for maximum correspondence, as measured using a sequence comparison algorithm (e.g., BLASTP and BLASTN or other algorithms available to persons of skill) or by visual inspection. Depending on the application, the percent homology can exist over a region of the sequence being compared, e.g., over a functional domain, or, alternatively, exist over the full length of the two sequences to be compared. By way of example, percent homology of a sequence may be compared to a reference sequence. For example, when using a sequence comparison algorithm, test and reference sequences may be input into a computer (and subsequence coordinates may be further designated if desired along with sequence algorithm program parameters). The sequence comparison algorithm then calculates the percent sequence identity or homology for the test sequence(s) relative to the reference sequence(s), based on the designated program parameters. Exemplary sequence alignment and/or homology algorithms are available through, Smith & Waterman, Adv. Appl. Math. 2:482 (1981), Needleman & Wunsch. J. Mol. Biol. 48:443 (1970). Pearson & Lipman, Proc. Nat'l. Acad. Sci. USA 85:2444 (1988), GAP, BESTFIT, FASTA, and TFASTA (in the Wisconsin Genetics Software Package, Genetics Computer Group, 575 Science Dr., Madison. Wis.), or by visual inspection (see generally Ausubel et al., infra). One example of an algorithm that is suitable for determining percent sequence identity and sequence similarity is the BLAST algorithm, which is described in Altschul et al., J. Mol. Biol. 215.403-410 (1990). Software for performing BLAST analyses is publicly available through the National Center for Biotechnology Information (www.ncbi.nlm.nih.gov/).

The present disclosure also pertains to methods of clinical diagnosis, prognosis, or theranosis of a subject performed by a medical professional using the methods disclosed herein. The methods, as described herein, can, for example, be performed by an individual, a health professional, or a third party, for example a service provider who interprets genotype information from the subject. As explained herein, a medical professional may initiate or modify treatment after receiving information regarding a diagnostic method of the present disclosure. For example, a medical professional may recommend a therapy or a change in therapy.

Antibodies of the instant disclosure can be used to isolate, detect and/or quantify IL-19 by standard techniques, such as affinity chromatography, immunoprecipitation, immunohistochemistry or ELISA-based assay. Such assay can be used to detect and/or evaluate the abundance and/or patterns of IL-19 expression for diagnostic, prognostic, or theranostic purposes to monitor polypeptide levels, for example in serum, plasma, blood or tissue as part of a clinical testing procedure, e.g., to determine the efficacy of a given treatment regimen.

IL-19 levels or measurements, as provided by assays of the present invention, may be absolute values (e.g., concentration within a biological sample) or relative values (e.g., concentration compared to a reference). As used herein, IL-19 is referred to as “increased” in a patient sample if the method for detecting IL-19 indicates that the level or concentration of IL-19 in the patient sample is higher than a reference value. Conversely. IL-19 is referred to as “decreased” in a patient sample if the IL-19 level or concentration of IL-19 in a patient sample is lower than a reference value, or for example, the IL-19 value measured in a previous patient sample.

A “reference value” as used herein refers to a known, or approximate concentration of IL-19 associated with a specific condition. The concentration levels in a reference value can be an absolute or relative amount, a range of amount, or a minimum amount, a mean amount, and/or a median amount of IL-19. A reference value can also serve as a baseline of IL-19 to which a value derived from a patient sample is compared. According to some embodiments, the reference value may include a reference value of approximately 21 pg/mL.

A “control standard.” as used herein, refers to a sample that can be used to compare the results obtained from a patient sample in the methods of the invention. Control standards can be cells, tissue, or known protein concentrations spiked into a media. The concentration levels in a control standard can be an absolute or relative amount, a range of amount, or a minimum amount, a mean amount, and/or a median amount of IL-19. A control standard can also serve as a baseline of IL-19 to which the patient sample is compared. The control standard can include a concentration value from the same patient or a known, normal reference of IL-19. According to some embodiments, the control standard may include a reference value of approximately 21 pg/mL. Further, in some embodiments, a control standard may express IL-19 concentrations in the form of a standard curve.

As used herein, the term “capture antibody” or “first antibody” refers to an IL-19 antibody capable of binding and capturing IL-19 in a patient sample under suitable conditions, such that the capture antibody-IL-19 complex can be separated from the rest of the sample. In some embodiments, the capture antibody is immobilized. In some embodiments, the capture antibody is labeled with a detectable label. In some embodiments, the capture antibody is immobilized in a “sandwich” immunoassay, and the capture or first IL-19 antibody binds a specific or first epitope region of IL-19. In such sandwich immunoassays, a “detection (or second) antibody” is also utilized. According to some embodiments a detection or second antibody may bind specifically to the capture antibody and may be labelled with a detectable label. In some embodiments, the detection of second antibody binds to the IL-19 already bound, or captured, by the capture or first antibody. In such embodiments, the detection antibody binds IL-19 at a second epitope region and may be labelled with a detectable label.

As understood in the art, an antibody of the present invention may be coupled to a “detectable label” to facilitate its detection As used herein, a detectable label is a moiety, composition or technique which can be used to detect the binding of the detection antibody to the capture antibody-IL-19 complex. According to some embodiments, the detectable label may be conjugated to the antibody (either capture or detection, as the case may be) directly or indirectly. Examples of detectable labels include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase: examples of suitable prosthetic group complexes include streptavidin/biotin and avidin/biotin; examples of suitable fluorescent materials include umbelliferone, fluorescein, fluorescein isothiocyanate, rhodamine, dichlorotnazinylamine fluorescein, dansyl chloride or phycoerythnn; an example of a luminescent material includes luminol; examples of bioluminescent materials include luciferase, luciferin, and aequorin, and examples of suitable radioactive material include ¹²⁵I, ¹³¹I, ³⁵S or ³H. Antibodies of the present invention can also be useful in pharmacogenomic analysis. Such embodiments, may be used to identify individuals that can benefit from specific or modified treatment modalities and/or monitor efficacy of present treatment regimens.

The term “diagnosis” or “diagnosing”, as used interchangeably herein, refers to methods by which the skilled artisan can estimate and/or determine the probability (“a likelihood”) of whether or not a patient is suffering from a given disease or condition. In the case of the present invention, “diagnosing” the patient includes using the results of an assay of the present invention to identify or diagnose an autoimmune disease or element related to an autoimmune disease and the patient (that is, the presence or occurrence of an autoimmune disease or the need for treatment, or the effectiveness of a treatment against the autoimmune disease with the patient). A diagnosis may, according to the present invention, be based on a combination of other clinical indicia, as understood by a healthcare professional, to arrive at a diagnosis.

### EXAMPLES

### Expression of IL-19 Antibodies

Murine-derived IL-19 antibodies of the present invention are generated employing hybridoma methodology (e.g., as as first described by Kohler et al., Nature, 256:495 (1975)). Briefly, the mouse is immunized with recombinant human IL-19 and lymphocytes capable of producing antibodies that bind human IL-19 are isolated and fused with a myeloma cell line using a suitable fusing agent for forming a hybridoma cell (Goding, Monoclonal Antibodies: Principles and Practice. pp. 59-103 (Academic Press, 1986)). Hybridomas are seeded and grown in a suitable culture medium (preferably containing one or more substances inhibiting survival of unfused myeloma cells). Binding specificity of monoclonal antibodies produced by hybridomas is then determined by by an in vitro binding assay (e.g., immunoprecipitation, radioimmunoassay (RIA), or enzyme-linked immunosorbent assay (ELISA)). Preferred hybridomas may be subcloned by limiting dilution procedures and grown by standard methods including in vivo as ascites tumors in an animal (Goding, Monoclonal Antibodies: Principles and Practice. pp. 59-103 (Academic Press, 1986)). Monoclonal antibodies secreted by the hybridomas (and or subclones) are purified according to conventional procedures such as, for example, affinity chromatography (e.g., protein A or protein G-Sepharose) or ion-exchange chromatography, hydroxylapatite chromatography, gel electrophoresis, dialysis, or the like. Affinity maturation of antibodies may be performed according to methods known in the field.

cDNA encoding antibodies of the present invention is sequenced using conventional procedures. cDNA sequences encoding the heavy and light chains may be cloned and engineered into a GS (glutamine synthetase) expression vector. The engineered immunoglobulin expression vector may then be stably transfected into CHO cells. As one of skill in the art will appreciate, mammalian expression of antibodies will result in glycosylation, typically at highly conserved N-glycosylation sites in the Fc region. Stable clones may be verified for expression of an antibody specifically binding to human IL-19. Positive clones may be expanded into serum-free culture medium for antibody production in bioreactors. Media, into which an antibody has been secreted, may be purified by conventional techniques. For example, the medium may be conveniently applied to a Protein A or G Sepharose FF column that has been equilibrated with a compatible buffer, such as phosphate buffered saline. The column is washed to remove nonspecific binding components. The bound antibody is eluted, for example, by pH gradient and antibody fractions are detected, such as by SDS-PAGE, and then pooled. The antibody may be concentrated and/or sterile filtered using common techniques. Soluble aggregate and multimers may be effectively removed by common techniques, including size exclusion, hydrophobic interaction, ion exchange, or hydroxyapatite chromatography. The product may be immediately frozen, for example at −70° C., or may be lyophilized. CDR sequences of exemplified embodiments of murine-derived IL-19 antibodies of the present invention, which have been affinity optimized as known in the art, are provided in Table 1.

TABLE 1
Murine Immunization-Derived Antibody CDR Amino Acid Sequences

| | Light Chain CDRs SEQ ID NOs. | | | Heavy Chain CDRs SEQ ID NOs. | | |
|---|---|---|---|---|---|---|
| Antibody | LCDR1 | LCDR2 | LCDR3 | HCDR1 | HCDR2 | HCDR3 |
| M_1 | 3 | 4 | 5 | 7 | 8 | 9 |
| M_2 | 11 | 12 | 13 | 15 | 16 | 17 |
| M_3 | 19 | 20 | 21 | 23 | 24 | 25 |
| M_4 | 27 | 28 | 29 | 31 | 32 | 33 |
| M_5 | 303 | 304 | 305 | 307 | 308 | 309 |

Rabbit-derived IL-19 antibodies of the present invention are generated after obtaining antibody gene sequences directly from B cells. Briefly, a rabbit is immunized with recombinant human IL-19 and mRNA is isolated from antigen-specific B cells enriched from PBMCs. Nucleic acid sequence encoding the heavy and light chain variable regions from this library are then cloned into a cell-based display system. Functional binding fragments are isolated from library, the individual gene sequences determined, cloned for recombinant IgG expression, and purified essentially as described above with regard to murine-derived IL-19 antibodies. CDR sequences of exemplified embodiments of rabbit-derived IL-19 antibodies of the present invention, which have been affinity optimized as known in the art, are provided in Table 2.

TABLE 2
Rabbitt Immunization-Derived Antibody CDR Amino Acid Sequences.

| | Light Chain CDRs SEQ ID NOs. | | | Heavy Chain CDRs SEQ ID NOs. | | |
|---|---|---|---|---|---|---|
| Antibody | LCDR1 | LCDR2 | LCDR3 | HCDR1 | HCDR2 | HCDR3 |
| R_1 | 35 | 36 | 37 | 39 | 40 | 41 |
| R_2 | 43 | 44 | 45 | 47 | 48 | 49 |
| R_3 | 51 | 52 | 53 | 55 | 56 | 57 |
| R_4 | 59 | 60 | 61 | 63 | 64 | 65 |
| R_5 | 90 | 91 | 92 | 93 | 94 | 95 |
| R_6 | 96 | 97 | 98 | 99 | 100 | 101 |
| R_7 | 102 | 103 | 104 | 105 | 106 | 107 |
| R_8 | 108 | 109 | 110 | 111 | 112 | 113 |
| R_9 | 114 | 115 | 116 | 117 | 118 | 119 |
| R_10 | 120 | 121 | 122 | 123 | 124 | 125 |
| R_11 | 126 | 127 | 128 | 129 | 130 | 131 |
| R_12 | 132 | 133 | 134 | 135 | 136 | 137 |
| R_13 | 138 | 139 | 140 | 141 | 142 | 143 |
| R_14 | 144 | 145 | 146 | 147 | 148 | 149 |
| R_15 | 150 | 151 | 152 | 153 | 154 | 155 |
| R_16 | 156 | 157 | 158 | 159 | 160 | 161 |
| R_17 | 162 | 163 | 164 | 165 | 166 | 167 |
| R_18 | 168 | 169 | 170 | 171 | 172 | 173 |
| R_19 | 174 | 175 | 176 | 177 | 178 | 179 |
| R_20 | 180 | 181 | 182 | 183 | 184 | 185 |
| R_21 | 186 | 187 | 188 | 189 | 190 | 191 |
| R_22 | 192 | 193 | 194 | 195 | 196 | 197 |
| R_23 | 198 | 199 | 200 | 201 | 202 | 203 |
| R_24 | 204 | 205 | 206 | 207 | 208 | 209 |
| R_25 | 210 | 211 | 212 | 213 | 214 | 215 |
| R_26 | 216 | 217 | 218 | 219 | 220 | 221 |
| R_27 | 222 | 223 | 224 | 225 | 226 | 227 |
| R_28 | 228 | 229 | 230 | 231 | 232 | 233 |
| R_29 | 234 | 235 | 236 | 237 | 238 | 239 |
| R_30 | 240 | 241 | 242 | 243 | 244 | 245 |
| R_31 | 246 | 247 | 248 | 249 | 250 | 251 |
| R_32 | 252 | 253 | 254 | 255 | 256 | 257 |
| R_33 | 258 | 259 | 260 | 261 | 262 | 263 |
| R_34 | 264 | 265 | 266 | 267 | 268 | 269 |
| R_35 | 270 | 271 | 272 | 273 | 274 | 275 |
| R_36 | 276 | 277 | 278 | 279 | 280 | 281 |
| R_37 | 282 | 283 | 284 | 285 | 286 | 287 |
| R_38 | 286 | 287 | 288 | 289 | 290 | 291 |
| R_39 | 295 | 296 | 297 | 299 | 300 | 301 |

Phage-derived IL-19 antibodies of the present invention are isolated from antibody phage libraries employing common techniques such as described above, as described in McCafferty et al., Nature, 348:552-554 (1990). Clackson et al., Nature, 352:624-628 (1991) and Marks et al., J. Mol. Biol., 222:581-597 (1991) cDNA sequences encoding the heavy and light chains of phage-derived antibodies of the present invention may be cloned and engineered into a GS (glutamine synthetase) expression vector for recombinant expression in a competent cell line, such as CHO cells. CDR sequences of exemplified embodiments of phage-derived IL-19 antibodies of the present invention are provided in Table 3.

TABLE 3
Phage-Derived Antibody CDR Amino Acid Sequences.

| | Light Chain CDRs SEQ ID NOs. | | | Heavy Chain CDRs SEQ ID NOs. | | |
|---|---|---|---|---|---|---|
| Antibody | LCDR1 | LCDR2 | LCDR3 | HCDR1 | HCDR2 | HCDR3 |
| P_1 | 67 | 68 | 69 | 71 | 72 | 73 |
| P_2 | 75 | 76 | 77 | 79 | 80 | 81 |
| P_3 | 83 | 84 | 85 | 87 | 88 | 89 |

### Binding Kinetics and Affinity

Bio-layer interferometry (BLI) assay, measured with a Octet Red96® instrument available from ForteBio (using HBS-EP+ running buffer (GE Healthcare, 10 mM Hepes pH7.4+150 mM NaCl+3 mM EDTA+0.05% surfactant P20) at 25° C.), is used to measure binding of the exemplified IL-19 antibodies of the present invention to recombinant human IL-19 (having the amino acid sequence set for in SEQ ID NO: 1).

Except as noted, all reagents and materials are from ForteBio (Freemont, CA). An AMQ biosensor is used to immobilize antibody of interest for analysis. Exemplified antibody samples of the present invention (R_1, R_2, R_3, R_4, R_39, M_1, M_2 and M_5) are prepared at 5 ug/mL by dilution into running buffer. Recombinant human IL-19 is prepared to concentrations of 270, 90, 30, 10, 3.33, 1.11, 0.370, and 0 (blank) nM by dilution into running buffer. Each analysis consists of: (1) capturing antibody samples on biosensors for 300 secs; (2) establishing a baseline by incubating antibody loaded biosensors with running buffer for 60 secs; (3) incubating antibody loaded biosensors with serially diluted recombinant human IL-19 for 300 secs to monitor association phase; (4) return of biosensor to running buffer to monitor dissociation phase.

Binding data is processed using standard double-referencing and fit to a 1:1 binding model using Data Analysis v9.0 evaluation software, to determine the association rate (kₒₙ, M⁻¹s⁻¹ units), dissociation rate (k_{off}, s⁻¹ units), and Rₘₐₓ (nm units). The equilibrium dissociation constant (K_{D}) was calculated from the relationship K_{D}=k_{off}/kₒₙ, and is in molar units. Results are provided in Table 4.

TABLE 4
SPR binding data to recombinant human IL-19.

| Exemplified | kₒₙ | k_{off} | K_{D}* |
|---|---|---|---|
| Antibody | (M⁻¹s⁻¹ units) | (M⁻¹s⁻¹ units) | (M) |
| R_1 | 3.33 × 10⁴ | 1.04 × 10⁻⁵ | 3.11 × 10⁻¹⁰ |
| R_2 | 4.28 × 10⁵ | 4.42 × 10⁻⁵ | 1.03 × 10⁻¹⁰ |
| R_3 | 3.91 × 10⁵ | <1.00 × 10⁻⁷ | <1.00 × 10⁻¹² |
| R_4 | 1.52 × 10⁵ | 1.30 × 10⁻³ | 8.52 × 10⁻⁹ |
| R_39 | 2.06 × 10⁵ | 3.48 × 10⁻² | 1.70 × 10⁻⁷ |
| M_1 | 1.10 × 10⁶ | 6.32 × 10⁻⁵ | 5.76 × 10⁻¹¹ |
| M_2 | 6.37 × 10⁴ | 1.85 × 10⁻⁴ | 2.90 × 10⁻⁹ |
| M_5 | 6.88 × 10⁵ | 3.58 × 10⁻⁵ | 5.21 × 10⁻¹¹ |

*K_{D} results are considered relative as the results are not normalized for influence of avidity.

### Epitope Mapping

PEPperCHIP® peptide microarray linear epitope mapping of exemplified antibody M_I against human IL-19 is performed, according to manufacturer instructions, for high resolution linear epitope mapping. Briefly, exemplified antibody M-1 is incubated with a custom PEPperCHIP® peptide microarray comprising overlapping 12-mer peptide fragments of human IL-19. Scanning intensity is resolved using manufacturer software. An epitope, consisting of residues 95-102 (EPNPKILR) of SEQ ID NO. 1 is revealed, according to PEPperCHIP® analysis, for exemplified antibody M_I. Other murine-derived and rabbit-derived exemplified antibodies of the present invention do not yield a linear epitope, indicating human IL-19 conformational epitope binding.

Hydrogen deuterium exchange coupled with mass spectrometry (HDX-MS) is performed to map epitope regions of human IL-19 recombinant protein for exemplified antibodies M_2, M_3, M_5 and R_39. Briefly, HDX-MS is performed on a Waters nanoACQUITY system with HDX technology, including a LEAP HDX robotic liquid handling system and mass analysis is performed on a Waters Xevo G2-Tof mass spectrometer. The complex of human IL-19 with exemplified antibodies M_2, M_3, M_5, and R_39 is prepared at the molar ratio of 1:1.2 in 10 mM sodium phosphate buffer, pH 7.4 containing 150 mM NaCl (1×PBS buffer). The deuterium exchange experiment is initiated adding 55 μL of D20 buffer containing 0.1×PBS to 5 ul of human IL-19 or the human IL-19/antibody complex at 15° C. for various amounts of time (0 s, 10 s, 1 min, 10 min, 60 min, 120/240 min). The reaction is quenched using equal volume of was 0.32M TCEP, 0.1M phosphate pH 2.5 for two minutes at 1° C. 50 μL of the quenched reaction is injected on to an on-line pepsin column (Waters BEH Enzymate) at 14° C., using 0.2% formic acid in water as the mobile phase at a flow rate of 100 μL/min for 4 min. The resulting peptic peptides are then separated on a C18 column (Waters, Acquity UPLC BEH C18, 1.7 μm, 1.0 mm×50 mm) fit with a Vanguard trap column using a 3 to 85% acetonitrile (containing 0.2% formic acid) gradient over 10 min at a flow rate of 50 μL/min. The separated peptides are directed into a Waters Xevo G2 time-of-flight (qTOF) mass spectrometer. The mass spectrometer is set to collect data in the MS^{E}, ESI⁺ mode; in a mass acquisition range of m/z 255.00-1950.00; with a scan time of 0.5 s. The Xevo G2 is calibrated with Glu-fibrinopeptide prior to use. All acquired data is mass corrected using a 2 μg/ml solution of LeuEnk in 50% ACN, 50% H₂O and 0.1% FA at a flowrate of 5 μl/min every 30 s (m z of 556.2771). The peptides are initially identified by Waters Protein Lynx Global Server 3.02. The processing parameters are set to low energy threshold at 100.0 counts, an elevated energy threshold at 50.0 counts and an intensity threshold at 1500.0 counts. The resulting peptide list is imported to Waters DynamX 3.0 software, with threshold of 5 ppm mass error, 20% fragments ions per peptide based on peptide length. The relative deuterium incorporation for each peptide is determined by processing the MS data for deuterated samples along with the non-deuterated control in DynamX.

Sequence coverage from 90.9 to 94.8% of human IL-19 protein, with HDX-MS as described, is observed. When in complex with the exemplified antibodies, decreased deuterium uptake is observed at the residues of SEQ ID NO. 1 as denoted: M_2 and M_3: residues 67-75 (QIIKPLDVC) and 125-136 (RQCHCRQEATNA); M_5 residues 90-100 (FKDHQEPNPKI); and R_39 residues 42-60 (QEIKRAIQAKDTFPNVTIL), 90-107 (FKDHQEPNPKILRKISSI), and 149-160 (VHAAAIKSLGEL).

### Binning Experiments

Binning experiments involve competing monoclonal antibodies against one another in a pairwise and combinatorial fashion for binding to a specific antigen. A “bin” is a relative concept, based upon the epitope regions represented within the panel of monoclonal antibodies being tested. Two antibodies belong to the same bin if they cannot pair with one another and share the same blocking profile when tested against the other antibodies (or bins of antibodies) in the test panel. Binning of exemplified antibodies of the present invention may be performed by cross-competition binding assays using the Octet Red96R), available from ForteBio, according to manufacturer instruction. Briefly, to determine if two antibodies share overlapping epitope regions, an exemplified antibody is labeled with biotin and captured onto streptavidin sensor tip. The coated biosensor tip is then incubated with recombinant human IL-19 to saturate the capture antibody binding sites. The capture antibody-antigen complex is then incubated with a detection antibody. A change in wavelength is detected if the detection antibody is capable of binding. Antibodies with a same binding profile are grouped together into the same bin. Results are presented in Table 5.

TABLE 5
Antibody Binning Groups

| Exemplified | Binning |
|---|---|
| Antibody | Group |
| M_1 | 1 |
| M_2 | 2 |
| M_3 | 2 |
| M_4 | 1 |
| M_5 | 1 |
| R_1 | 3 |
| R_2 | 3 |
| R_3 | 3 |
| R_4 | 4 |

### Neutralization of IL-19 In Vitro

Antibodies of the present invention are expected to neutralize IL-19. Neutralization of IL-19 activity by antibodies of the present invention may be assessed by one or more of the IL-19/IL-19 receptor binding assay formats, as well as IL-19 binding assays, for example, as described below.

In an example, IL-19 is radiolabeled, for example, with iodine-125 or tritium. Cells (e.g., transfected with the IL-19 receptor, transformed keratinocytes that endogenously express the IL-19 receptor, or primary human cells such as keratinocytes that express the IL-19 receptor) expressing the IL-19 receptor such as IL-20R1 are used in the assay which may be conducted in buffered media, such as HBSS with calcium and magnesium and with whole cells. Accordingly, the cells may be incubated with the labeled IL-19 in the assay buffer at 4, 20 or 37° C. for 1 to 6 hours. A readout provides the amount of label bound to the cells after separation of unbound tracer, such as with filtration though a glass fiber filter. Alternatively, neutralization may be assessed by way of a proximity based assay, such as with SPA beads. Further, a neutralization assay utilizing non-radioactive label IL-19 protein may be used.

Such neutralization assays involve pre-incubation of the antibody being assessed with the labeled IL-19 (for example, for 1 hour) before addition to the binding assay (as well as control samples in which no antibody targeting IL-19 is involved). Concentrations of labeled IL-19 near the 50% binding level (EC50) may be used, as well as varying concentrations (for example, in assessing a dose response of the antibody such as from about 100 micromolar down to about 1 picomolar). Antibody inhibition assessed for a range allows for determination of potency (IC50).

According to another method for assessing neutralization of IL-19 by antibodies of the present invention, the IL-19 protein is labeled with a fluorescent dye for flow cytometry (e.g., Alexa-647) and used to label cells, such as human keratinocytes. The binding may then be measured using flow cytometry. Neutralization of IL-19 by the antibody is assessed by pre-incubating the antibody with the labeled IL-19 (for example, for 1 hour at 4° C.) before adding the mixture to the cells (with staining occurring for about 3 hours at 4° C.). Concentrations of fluorescently labeled IL-19 near its 50% binding level (EC50) may used, as well as varying concentrations (for example, in assessing a dose response of the antibody such as from about 100 micromolar down to about 1 picomolar). Antibody inhibition of binding of the labeled IL-19 to its receptor is reflected by measurement of loss of labeled cells, and a potency (IC50) for the antibody may be determined.

Alternatively, a biophysical assay such as bio-layer interferometry (BLI) may be used for assessing neutralization of IL-19 by antibodies of the present invention. Binding between a ligand immobilized on the biosensor tip surface and an analyte in solution produces an increase in optical thickness at the biosensor tip, which results in a wavelength shift (expressed in nm). According to such assay, the IL-19 receptor (i.e., IL-20R1) is expressed in a membrane-free manner (such as with an Fc-fusion e.g. IL20Rbeta Fc chimera protein from R&D Systems catalog 1788-IR-050). AMQ or anti-rabbit conjugated biosensors (ForteBio) are used to immobilize anti-IL-19 antibody of interst (M_1, M_2, M_3, M_5 and R_39). The immobilized antibodies are then incubated with recombinant human IL-19 protein diluted to 100 nM using HBS-EP+ running buffer (GE Healthcare, 10 mM Hepes pH7.4+150 mM NaCl+3 mM EDTA+0.05% surfactant P20) for 240-300 secs. The human IL-19, after binding to the anti-IL-19 antibody, is assessed by incubation with human IL-20R beta Fc-fusion protein for 240-300 secs. The ability of the antibody to block or neutralize binding of the IL-19 ligand to the soluble receptor is observed as a minimal (<0.025 nm) increase in the wavelength during this step of the assay. Results are provided inTable 6.

TABLE 6
In vitro neutralization.

| | IL-20R beta Fc | |
|---|---|---|
| Exemplified | protein binding | |
| Antibody | response (nm) | Neutralization |
| M_1 | −0.0075 | Yes |
| M_2 | 0.3614 | No |
| M_3 | 0.3539 | No |
| M_5 | −0.0124 | Yes |
| R_39 | −0.0421 | Yes |

Another method of assessing neutralization of IL-19 by antibodies of the present invention includes addition of such antibody, pre-incubated with human IL-19, to human keratinocytes. Exogenous IL-19 to human keratinocytes induces expression of additional IL-19 and other inflammatory molecules such as IL-8, CCL20 and S100A7. After pre-incubation of the antibody and IL-19 (for example, for 1 hr at 4° C.), the antibody-IL-19 mixture is added to cultured human keratinocytes. The cells are then cultured for 1 to 48 hrs and one or more of IL-19, IL-8, CCL20 and S100A7 (or other molecule expressed downstream of IL-19) in the supernatant is measured, for example, by ELISA (alternatively, mRNA of the downstream molecule may be measured). Antibody inhibited IL-19 function will demonstrate a reduced expression of the downstream molecule by the cultured keratinocytes.

PathHunter® eXpress IL20RA/IL20RB dimerization assay (DiscoverX product code 93-1027E3) is used to assess ability of exemplified antibodies to prevent binding of human IL-19 recombinant protein in a cell-based assay format. The assay detects ligand induced dimerization of two subunits of a receptor-dimer pair. The cells have been engineered to co-express one receptor subunit fused to enzyme donor and a second dimer partner fused to enzyme acceptor. Binding of an agonist to one receptor subunit induces it to interact with its dimer partner, forcing complementation of the two enzyme fragments resulting in the formation of a functional enzyme that hydrolizes a substrate to generate a chemiluminescent signal. Briefly, cells are plated at 2500 cells per well and cultured at 37° C./5% CO₂ for 4 hours before addition of human IL-19 recombinant protein pre-mixed with various concentrations, ranging from 10 to 0.00001 μg/ml including a buffer only control, of exemplified antibodies (M_1 and M_5). Human IL-19 recombinant protein with and without exemplified antibodies mixture is then incubated with cells overnight at 37° C./5% CO₂. Substrate buffer is added to cells and incubated at room temperature for 1 hour in the dark before luminescent detection. The concentration of exemplified antibody resulting in inhibition of 50% of signal (IC50) and the maximum signal inhibition percentage (% inhibition) is tabulated for eight experiments with standard error of the mean (SEM) denoted below. Results are provided in Table 7.

TABLE 7
Cell-based neutralization.

| Exemplified | IC50 +/− SEM | |
|---|---|---|
| Antibody | (μg/ml) | % inhibition +/− SEM |
| M_1 | 0.48 +/− 0.06 | 98.59 +/− 0.42 |
| M_5 | 0.64 +/− 0.20 | 105.03 +/− 1.38 |

### IL-19 Assay

Plaque type psoriasis is currently measured based on measures of overall body surface involvement (BSA) and/or assessments of degree of erythema, thickness and scale of psoriasis lesions (PASI). However, given subjective input required with these methods they may not be linear depending on severity of skin involvement. No single blood-derived marker has been identified which allows for assessing overall psoriasis activity. Therefore, a more objective and reproducible method to determine severity is desired. The present invention provides a highly sensitive and specific assay to measure IL-19 levels in patients samples such as blood, serum and plasma. As illustrated herein, the IL-19 assay of the present invention provides an accurate diagnostic tool for therapy responsiveness (i.e., a predictive biomarker), disease reoccurrence (i.e., a prognostic biomarker), disease onset, and disease severity in patients with moderate-to-severe Ps0.

According to an exemplified embodiment, a sandwich ELISA assay for the sensitive detection of IL-19 is provided herein. The assay utilizes exemplified IL-19 antibodies of the present invention, for example, as set forth in Table 1, 2 or 3. According to an exemplified embodiment, a first IL-19 antibody (selected from Table 1, 2 or 3) is utilized as an IL-19 capture antibody and a second IL-19 antibody (selected from Table 1, 2 or 3) is utilized as an IL-19 reporter antibody. In some embodiments the first and second IL-19 antibodies are selected from separate epitope bins (for example, in specific embodiments, exemplified IL-19 antibody M_I is paired with exemplified IL-19 antibody M_2). According to some embodiments, one milligram of the first IL-19 antibody (the capture antibody) is biotinylated using Pierce biotinylation kit (Cat #) and one milligram of the second IL-19 antibody (the reporter antibody) is labeled with ruthenium using MesoScale Discovery (MSD) kit for electrochemiluminescent (ECL) detection. According to such embodiment, labeled antibodies are evaluated using MALDI-TOF to ensure suitable labeling, and then diluted in 50% glycerol and stored at −20° C. prior to use.

Streptavidin-coated 96-well MSD plates are washed three times with TBST (Tris buffered saline containing 10 mmol/L Tris pH 7.40, 150 mmol/L NaCl with 1 mL Tween 20/L) and then blocked with TBS-T plus 1% BSA for 1 hour at room temperature. Plates are again washed and wells are then incubated with biotinylated IL-19 capture antibody (1 mg/L) for 1 hour. Thereafter, plates are again washed prior to patient sample testing.

During patient sample testing, a standard curve is generated using 50 μL of recombinant human IL-19 control standard (serially diluted IL-19 recombinant protein ranging from 100-0.0001 ng/L, and including a zero blank, in assay buffer of 50 mmol/L HEPES, pH 7.40, 150 mmol/L NaCl, 10 mL/L Triton X-100, 5 mmol/L EDTA, and 5 mmol/L EGTA). Data from ten separate standard curves, prepared as described herein, shows a dynamic range of 10-1 pg/mL to 105 μg/mL of IL-19 (providing an sensitive and broad dynamic range in the therapeutic and diagnostic assays provided herein). Patient samples (which, according to the present invention, may include blood, serum or plasma) are diluted 1:4 in assay buffer and added to respective wells. The plate is incubated overnight at 4° C. Following incubation, wells are aspirated and washed 3 times with TBST. Thereafter, 50 L of ruthenium-labeled IL-19 detection antibody (0.5 mg/L) is added to the wells for a 1-hour incubation at room temperature. Following incubation, wells are aspirated and washed 3 times with TBST. Thereafter, 150 μl of 2×MSD read buffer is added. Ruthenium electrochemiluminescence in the wells is detected using a MSD Sector 6000 plate reader. Data is analyzed and IL-19 MSD immunoassay calibration curve fitting is performed using MesoScale Discovery software. SAS® software version 9.4 (PROC MIXED) is used for assessing treatment effects on IL-19 levels using a mixed effects model with an unstructured covariance matrix and log₁₀ transformed IL-19 concentrations (SAS. Version 9.4 for UNIX; SAS Institute Inc.: Cary, NC, 2016). Statistical analysis is generated with the ggplot and pROC packages using R version 3.3.3 statistical computing environment (www.R-project.org, Vienna, Austria, 2017).

### IL-19 Serum Concentrations in Ps0 Patients Versus Healthy Groups

A study of IL-19 levels in serum of 125 Ps0 patients, pre-treatment, were compared to IL-19 serum levels of 36 healthy volunteer samples. Using an IL-19 assay essentially as described above, with a capture antibody selected from bin 1 (specifically, exemplified IL-19 antibody M_1) and a detection antibody selected from bin 2 (specifically, exemplified IL-19 antibody M_2), serum IL-19 concentrations (pg/ml) of each patient sample is measured. The geometric mean of serum IL-19 concentrations for healthy volunteers (n=36) is measured at 11 pg/mL (with a range of 4 to 51 μg/mL, and a 95% confidence level at less than 21 pg/mL) whereas the geometric mean of serum IL-19 concentrations for Ps0 patients (pre-treatment) (n=112) is measured at 87 pg/mL. Thus, the present invention provides an IL-19 blood-based assay allowing for the diagnosis of Ps0 patients.

### Ps0 Study in Anti-IL-17 Treatment Groups

A study of IL-19 levels, in serum of 125 Ps0 patients treated with a therapeutic antibody targeting IL-17, ixekizumab, is performed. The study includes five treatment group doses of: 10 mg (n=24), 25 mg (n=23), 75 mg (n=26), or 150 mg (n=28) of ixekizumab or placebo (n=24). Administration of all treatment doses is subcutaneous, and doses are administered starting at week 0 and every 2 weeks thereafter up to week 16 (inclusive). Using an IL-19 assay as described above, with a capture antibody selected from bin 1 (specifically, exemplified IL-19 antibody M_1]) and a detection antibody selected from bin 5 (specifically, exemplified IL-19 antibody M_2), serum IL-19 concentrations (pg/ml) of each patient is measured at week 0 (pre-treatment dosing), week 2 and week 12. Serum IL-19 concentration levels, percent PASI change and PASI 75 response are provided in Tables 8-10.

As shown, 36 out of 41 (87.8%) patients with a greater than or equal to 5-fold reduction in IL-19 from week 0 to week 2 achieved PASI 75 or greater by week 16 (whereas only 24 of 56 (42.9%) patients with less than a 5-fold reduction in IL19 from week 0 to week 2 achieved a PASI 75 response by week 16. Further, 37 out of 42 (88%) patients with a greater than or equal to 5-fold reduction in IL-19 from week 0 to week 9 achieved PASI 75 or greater by week 12 (whereas only 22 out of 53 (41.5%) patients with less than a 5-fold reduction in IL19 from week 0 to week 16 achieved a PASI 75 response by week 16 (data reflects a drop-out of 2 patients between weeks 2 and 12). For the placebo treatment group, no significant change in IL-19 concentrations were observed during the 16-week trial period.

TABLE 8
IL-19 serum levels (geometric mean) per treatment group.

| Treatment | Week 0 | Week 2 | Week 16 |
|---|---|---|---|
| Group | (baseline IL-19 pg/mL) | (IL-19 pg/mL) | (IL-19 pg/mL) |
| 150 | 87.1 | 13.9 | 11.9 |
| (n = 28) | | | |
| 75 | 86.7 | 11.6 | 9.4 |
| (n = 26) | | | |
| 25 | 89.0 | 22.3 | 13.6 |
| (n = 23) | | | |
| 10 | 111.6 | 45.3 | 57.3 |
| (n = 24) | | | |
| Placebo | 67.4 | 66.7 | 57.2 |
| (n = 24) | | | |

Table 8 provides data showing IL-19 measured in treatment groups over 16 weeks of treatment with placebo or various ixekizumab doses.

TABLE 9
Patients achieving at least PASI 75 at week 16 per treatment group.

| | Week 2 |
|---|---|
| Treatment | (number of patients achieving |
| Group | at least PASI 75) |
| 150 | 22 |
| (n = 24) | |
| 75 | 21 |
| (n = 24) | |
| 25 | 16 |
| (n = 20) | |
| 10 | 5 |
| (n = 19) | |
| Placebo | 1 |
| (n = 20) | |

Table 9 provides the the number of patients, per treatment group, achieving PASI 75 by week 16.

TABLE 10
Assessment of serum IL-19 concentration and PASI at week 16.

| | | % patients with | % patients with |
|---|---|---|---|
| | | serum IL-19 | serum IL-19 |
| | % of patients | concentration ≤ | concentration ≤ |
| | with serum IL-19 | 21 pg/mL | 21 pg/mL not |
| Treatment | concentration ≤ | achieving at | achieving at |
| Group | 21 pg/mL | least PASI 75 | least PASI 75 |
| 150 | 92% (23/25) | 95% (19/20) | 5% (1/20) |
| (n = 25) | | | |
| 75 | 91.7% (22/24) | 86.4% (19/22) | 13.6% (3/22) |
| (n = 24) | | | |
| 25 | 70% (14/20) | 92.9% (13/14) | 7.1% (1/14) |
| (n = 20) | | | |
| 10 | 42.1% (8/19) | 62.5% (5/8) | 37.5% (3/8) |
| (n = 19) | | | |
| Placebo | 25% (5/20) | 20% (1/5) | 80% (4/5) |
| (n = 20) | | | |

Table 10 presents correlations of IL-19 serum levels and PASI in psoriasis patients after 16 weeks of placebo or various ixekizumab treatments (21 ng/L indicates the upper limit of the normal range of IL-19 in healthy subjects). It was noted that PASI 100 improvements at 16 weeks were preceded by reduction of circulating IL-19 to near normal concentrations after 2 weeks of treatment.

The data provided in Tables 8-10 demonstrate that the IL-19 assay of the present invention provides a valuable tool for diagnosis and therapeutic prognostication of Ps0 patients treated with IL-17 antibodies.

### Ps0 Study in Anti-TNFα Treatment Groups

A study of IL-19 levels, in serum of 35 Ps0 patients that were complete responders when treated with the FDA approved TNFα antagonist, entanercept, is performed. Treatment groups of 50 mg of etanercept (n=35) administered biweekly or placebo are compared. Administration of both treatment groups is subcutaneous. Using an IL-19 assay as described above, with a capture antibody selected from bin 1 (specifically, exemplified IL-19 antibody M_1) and a detection antibody selected from bin 2 (specifically, exemplified IL-19 antibody M_2), serum IL-19 concentrations (pg/ml) of each patient is measured at week 0 (pre-treatment dosing), week 1, week 4 and week 12. IL-19 levels are assessed for prognostic value with PASI improvement at weeks 4 and 12. Serum IL-19 concentration levels are presented in Table 11; prognostic values at week 4 showing correlations of IL-19 serum levels and PASI in psoriasis patients (21 ng/L indicates the upper limit of the normal range of IL-19 in healthy subjects) is presented in Table 12; prognostic values at week 12 P showing correlations of IL-19 serum levels and PASI in psoriasis patients (21 ng/L indicates the upper limit of the normal range of IL-19 in healthy subjects) is presented in Table 13.

TABLE 11
IL-19 (pg/mL) serum levels (geometric mean) per treatment group.

| Treatment | Week 0 | | | |
|---|---|---|---|---|
| Group | (baseline) | Week 1 | Week 4 | Week 12 |
| Etanercept | 98.2 | 42.3 | 24.3 | 14.2 |
| (n = 35) | | | | |

TABLE 12
Assessment of serum IL-19 concentration and PASI at week 4.

| | | % patients with | % patients with |
|---|---|---|---|
| | | serum IL-19 | serum IL-19 |
| | % of patients | concentration ≤ | concentration ≤ |
| | with serum IL-19 | 21 pg/mL | 21 pg/mL not |
| Treatment | concentration ≤ | achieving at | achieving at |
| Group | 21 pg/mL | least PASI 75 | least PASI 75 |
| Etanercept | 37.9% (44/116) | 22.7% (10/44) | 77.3% (34/44) |
| (n = 160) | | | |

TABLE 13
Assessment of serum IL-19 concentration and PASI at week 12.

| | | % patients with | % patients with |
|---|---|---|---|
| | | serum IL-19 | serum IL-19 |
| | % of patients | concentration ≤ | concentration ≤ |
| | with serum IL-19 | 21 pg/mL | 21 pg/mL not |
| Treatment | concentration ≤ | achieving at | achieving at |
| Group | 21 pg/mL | least PASI 75 | least PASI 75 |
| Etanercept | 42.9% (69/161) | 49.3% (34/69) | 50.7% (35/69) |
| (n = 161) | | | |

As shown above, on average TNF antagonist-treated patients experienced a median reduction in serum IL-19 of greater than 40 (pg/mL) after 1 week and a median reduction in serum IL-19 of grater than 70 (pg/mL) at week 4. The data provided in Tables 11-13 demonstrate that the IL-19 assay of the present invention provides a valuable tool for diagnosis and therapeutic prognostication of Ps0 patients treated with TNFα antibodies.

### Ps0 Study in Anti-IL-23 Treatment Groups

A study of IL-19 levels, in serum of Ps0 patients treated with a therapeutic antibody targeting IL-23, mirikizumab, is performed. Eight treatment groups of: 5 mg, 20 mg, 60 mg, 120 mg, 200 mg, 350 mg, and 600 mg of mirikizumab, or placebo, are assessed. Administration of each treatment group, as a single subcutaneous dose, occurs at day 0. Using an IL-19 assay as described above, with a capture antibody selected from bin 1 (specifically, exemplified IL-19 antibody M_1) and a detection antibody selected from bin 2 (specifically, exemplified IL-19 antibody M_2), serum IL-19 concentrations (pg/ml) of each patient is measured between visits 2 and 12. IL-19 levels are also assessed for prognostic value with PASI improvement between visits 2 and 12. Serum IL-19 concentration levels are presented in Table 14; a comparative of serum levels at week 8 is presented in Table 15; and prognostic values at week 8 showing correlations of IL-19 serum levels and PASI in psoriasis patients (21 ng/L indicates the upper limit of the normal range of IL-19 in healthy subjects) are presented in Table 16. Decreases in serum IL-19 correlated with improvement in PASI score demonstrating the IL-19 assay of the present invention provides a valuable tool for diagnosis and therapeutic prognostication of Ps0 patients treated with IL-23 antibodies.

TABLE 14
IL-19 (geometric mean) serum levels per treatment group.

| Treatment | Day 1 | | | | |
|---|---|---|---|---|---|
| Group | (baseline) | Day 15 | Day 29 | Day 57 | Day 71 |
| 5 | 16.2 | 13.9 | 10.4 | 13.2 | 14.5 |
| (n = 5) | | | | | |
| 20 | 37.2 | 27.2 | 25.4 | 33.5 | 28.4 |
| (n = 5) | | | | | |
| 60 | 49.8 | 9.0 | 10.8 | 11.9 | 10.9 |
| (n = 5) | | | | | |
| 120 | 52.4 | 26.6 | 25.2 | 22.2 | 18.2 |
| (n = 5) | | | | | |
| 200 | 69.6 | 36.0 | 23.3 | 21.9 | 22.5 |
| (n = 5) | | | | | |
| 350 | 58.4 | 35.2 | 22.4 | 23.7. | 18.0 |
| (n = 5) | | | | | |
| 600 | 57.3 | 30.1 | 21.8 | 14.4. | 15.8 |
| (n = 5) | | | | | |
| placebo | 58.9 | 52.2 | 47.0 | 40.4. | 47.1 |
| (n = 7) | | | | | |

TABLE 15
IL-19 (geometric mean) serum levels per treatment group.

| Treatment | Week 0 | Week 8 |
|---|---|---|
| Group | (Baseline) | (End of induction) |
| LY 300 mg | 106.5 | 18.2 |
| (n = 50) | | |
| Placebo | 158.0 | 154.9 |
| (n = 52) | | |

TABLE 16
Assessment of serum IL-19 concentration and PASI at week 8.

| | | % patients with | % patients with |
|---|---|---|---|
| | | serum IL-19 | serum IL-19 |
| | % of patients | concentration ≤ | concentration ≤ |
| | with serum IL-19 | 21 pg/mL | 21 pg/mL not |
| Treatment | concentration ≤ | achieving at | achieving at |
| Group | 21 pg/mL | least PASI 75 | least PASI 75 |
| LY 300 mg | 58.3% (28/48) | 71.4% (20/28) | 28.6% (8/28) |
| (n = 48) | | | |
| Placebo | 13.7% (7/51) | 28.6% (2/7) | 71.4% (5/7) |
| (n = 51) | | | |

### Ps0 Study in JAK1 and JAK2 Kinase Inhibitor Treatment Groups

A study of IL-19 levels, in serum of Ps0 patients treated with the therapeutic selective JAK1 and JAK2 inhibitor, baricitinib, is performed. Treatment groups of 2 mg, 4 mg, 8 mg, and 10 mg of baricitinib, or placebo, are assessed. Each treatment group is 10 orally administered once daily. Using an IL-19 assay as described above, with a capture antibody selected from bin 1 (specifically, exemplified IL-19 antibody M_1) and a detection antibody selected from bin 2 (specifically, exemplified IL-19 antibody M_2), serum IL-19 concentrations (pg/ml) of each patient is measured at baseline and following each treatment. IL-19 levels are also assessed for prognostic value with PASI improvement. Serum IL-19 concentration levels are presented in Table 17. PASI prognostic data (at week 12) showing correlations of IL-19 serum levels and PASI in psoriasis patients (21 ng/L indicates the upper limit of the normal range of IL-19 in healthy subjects) are presented in Table 18. The data demonstrates a decreases in serum IL-19 correlates with improvement in PASI score demonstrating the IL-19 assay of the present invention provides a valuable tool for diagnosis and therapeutic prognostication of Ps0 patients treated with JAK1 and JAK2 inhibitors.

TABLE 17
IL-19 serum levels (geometric mean) per treatment group.

| Treatment | Week 0 | | |
|---|---|---|---|
| Group | (baseline) | Week 2 | Week 12 |
| 2 | 182.3 | 98.5 | 64.8 |
| (n = 32) | | | |
| 4 | 134.9 | 70.2 | 60.1 |
| (n = 72) | | | |
| 8 | 177.3 | 67.0 | 47.5 |
| (n = 64) | | | |
| 10 | 110.1 | 39.7 | 25.7 |
| (n = 69) | | | |
| placebo | 116.9 | 131.8 | 95.3 |
| (n = 34) | | | |

TABLE 18
Assessment of serum IL-19 concentration and PASI at week 12.

| | | % patients with | % patients with |
|---|---|---|---|
| | | serum IL-19 | serum IL-19 |
| | % of patients | concentration ≤ | concentration ≤ |
| | with serum IL-19 | 21 pg/mL | 21 pg/mL not |
| Treatment | concentration ≤ | achieving at | achieving at |
| Group | 21 pg/mL | least PASI 75 | least PASI 75 |
| 2 | 13.8% (4/29) | 50% (2/4) | 50% (2/4) |
| (n = 29) | | | |
| 4 | 31.8% (21/66) | 47.6% (10/21) | 52.4% (11/21) |
| (n = 66) | | | |
| 8 | 38.9% (21/54) | 85.7% (18/21) | 14.3% (3/21) |
| (n = 54) | | | |
| 10 | 46.6% (27/58) | 77.8% (21/27) | 22.2% (6/27) |
| (n = 58) | | | |
| Placebo | 14.8% (4/27) | 100% (4/4) | 0% (0/4) |
| (n = 27) | | | |

### AD Study in JAK1 and JAK2 Kinase Inhibitor Treatment Groups

A study of IL-19 levels, in serum of 123 patients with moderate-to-sever atopic dermatitis treated with the therapeutic selective JAK1 and JAK2 inhibitor, baricitinib, is performed. Treatment groups of 2 mg and 4 mg of baricitinib, or placebo, are compared. Each treatment group is orally administered once daily. Using an IL-19 assay as described above, with a capture antibody selected from bin 1 (specifically, exemplified IL-19 antibody M_1) and a detection antibody selected from bin 2 (specifically, exemplified IL-19 antibody M_2), serum IL-19 concentrations (pg/ml) of each patient is measured at baseline (pre-treatment) and at weeks 4 and 16. IL-19 levels are assessed for prognostic value with EASI score improvement. Serum IL-19 concentration levels are presented in Table 19; EASI prognostic values (at week 16) showing correlations of IL-19 serum levels in AD patients (21 ng/L indicates the upper limit of the normal range of IL-19 in healthy subjects) are presented in Table 20. The data demonstrates baseline IL-19 concentrations in AD patients were found to be elevated compared to normal (geometric mean of 34 pg/mL in AD patients). The data also demonstrates a decreases in serum IL-19 at weeks 4 and 16 correlates with improvement in EASI score at week 16 demonstrating the IL-19 assay of the present invention provides a valuable tool for diagnosis and therapeutic prognostication of Ps0 patients treated with JAK1 and JAK2 inhibitors.

TABLE 19
IL-19 (geometric mean) serum levels per treatment group.

| Treatment | Week 0 | | |
|---|---|---|---|
| Group | (baseline) | Week 4 | Week 16 |
| 2 | 27.8 | 18.8 | 25.1 |
| (n = 37) | | | |
| 4 | 30.3 | 18.0 | 21.1 |
| (n = 38) | | | |
| placebo | 44.4 | 27.5 | 23.6 |
| (n = 49) | | | |

TABLE 20
Assessment of serum IL-19 concentration and EASI at week 16.

| | | % patients with | % patients with |
|---|---|---|---|
| | | serum IL-19 | serum IL-19 |
| | % of patients | concentration ≤ | concentration ≤ |
| | with serum IL-19 | 21 pg/mL | 21 pg/mL not |
| Treatment | concentration ≤ | achieving at | achieving at |
| Group | 21 pg/mL | least EASI 75 | least EASI 75 |
| 2 | 51.9% (14/27) | 64.3% (9/14) | 35.7% (5/14) |
| (n = 27) | | | |
| 4 | 63.0% (17/27) | 58.8% (10/17) | 41.2% (7/17) |
| (n = 27) | | | |
| Placebo | 50% (14/28) | 64.3% (9/14) | 35.7% (5/14) |
| (n = 28) | | | |

### IL-19 Serum Concentrations in Renal Failure and Diabetes Patient Groups

IL-19 levels are measured in healthy donors (n=20), renal failure patients (n=16), diabetes patients without renal failure (n=20), and diabetes patients with renal failure (n=21). Using an IL-19 assay as described above, with a capture antibody selected from bin 1 (specifically, exemplified IL-19 antibody M_1) and a detection antibody selected from bin 2 (specifically, exemplified IL-19 antibody M_2), baseline serum IL-19 concentrations (pg/ml) of each patient is measured. The data demonstrates markedly elevated IL-19 levels in renal failure patients (40±6 pg/mL), diabetes patients without renal failure (17±3 pg/mL), and diabetes patients with renal failure (46±9 pg/mL) as compared to healthy donors (8±1 pg/mL) IL-19 levels. Thus, the IL-19 assay of the present invention provides a valuable tool for diagnosis and therapeutic prognostication of diabetes and renal failure patients.

### PsA Study in Anti-IL-17 Treatment Groups

A study of IL-19 levels, in serum of 309 PsA patients treated with a therapeutic antibody targeting IL-17, ixekizumab, is performed. The study includes three treatment groups: (i.) anti-IL-17 treatment group, administered ixekizumab 160 mg at baseline followed by an 80 mg dose administered every two weeks thereafter for 12 weeks (n=103); (ii.) anti-IL-17 treatment group, administered ixekizumab 160 mg at baseline followed by an 80 mg dose administered every four weeks thereafter for 12 weeks (n=107); or (iii.) placebo (n-105). Administration of all treatment doses is subcutaneous. Using an IL-19 assay as described above, with a capture antibody selected from bin 1 (specifically, exemplified IL-19 antibody M_1) and a detection antibody selected from bin 2 (specifically, exemplified IL-19 antibody M_2), serum IL-19 concentrations (pg/ml) of each patient is measured at week 0 (pre-treatment dosing), week 4 and week 12. Serum IL-19 concentration levels are presented in Table 21; week 4 and 12 PASI prognostic values showing correlations of IL-19 serum levels and PASI in patients (21 ng/L indicates the upper limit of the normal range of IL-19 in healthy subjects) are presented in Tables 22 and 23, respectively.

TABLE 21
IL-19 (geometric mean) serum levels per treatment group.

| Treatment | Week 0 | | |
|---|---|---|---|
| Group | (baseline) | Week 4 | Week 12 |
| Ixe | 23.0 | 6.0 | 6.1 |
| Q2W | | | |
| (n = 103) | | | |
| Ixe | 31.3 | 7.6 | 7.3 |
| Q4W | | | |
| (n = 107) | | | |
| placebo | 23.7 | 27.0 | 28.4 |
| (n = 105) | | | |

TABLE 22
Assessment of serum IL-19 concentration
at week 4 and PASI at week 12.

| | | % patients with | % patients with |
|---|---|---|---|
| | | serum IL-19 | serum IL-19 |
| | % of patients | concentration ≤ | concentration ≤ |
| | with serum IL-19 | 21 pg/mL | 21 pg/mL not |
| Treatment | concentration ≤ | achieving at | achieving at |
| Group | 21 pg/mL | least PASI 75 | least PASI 75 |
| Ixe | 97.5% (79/81) | 72.2% (57/79) | 27.8% (22/79) |
| Q2W | | | |
| (n = 81) | | | |
| Ixe | 90.8% (79/87) | 73.4% (58/79) | 26.6% (21/79) |
| Q4W | | | |
| (n = 87) | | | |
| Placebo | 46.4% (39/84) | 20.5% (8/39) | 79.5% (31/39) |
| (n = 84) | | | |

TABLE 23
Assessment of serum IL-19 concentration
at week 12 and PASI at week 12.

| | | % patients with | % patients with |
|---|---|---|---|
| | | serum IL-19 | serum IL-19 |
| | % of patients | concentration ≤ | concentration ≤ |
| | with serum IL-19 | 21 pg/mL | 21 pg/mL not |
| Treatment | concentration ≤ | achieving at | achieving at |
| Group | 21 pg/mL | least PASI 75 | least PASI 75 |
| Ixe | 96.7% (87/90) | 73.1% (57/78) | 26.9% (21/78) |
| Q2W | | | |
| (n = 90) | | | |
| Ixe | 93.5% (87/93) | 74.1% (60/81) | 25.9% (21/81) |
| Q4W | | | |
| (n = 93) | | | |
| Placebo | 43.0% (37/86) | 24.3% (9/37) | 75.7% (28/37) |
| (n = 86) | | | |

Table 21 shows baseline IL-19 levels in psoriatic arthritis patients are increased compared to the reference value of healthy volunteers (represented by the shaded grey region). Placebo treatment does not result in significant change in IL-19 over the 12-week time study. However, both ixekizumab treatment groups show lowering of IL-19 to near normal levels after 4 weeks; lowering which is sustained over the 12 week treatment. Tables 22 and 23 show the relationship between IL-19 levels in PsA patients after either 4 or 12 weeks of placebo or ixekizumab treatment groups and the PASI score at 12 weeks. PASI 100 improvements at 12 weeks were correlated with a reduction of circulating IL-19 concentrations to near normal levels, with the majority of the poor PASI responders being in the placebo group. The data provided in Tables 21-23 demonstrate that the IL-19 assay of the present invention provides a valuable tool for diagnosis and therapeutic prognostication of PsA patients treated with IL-17 antibodies.

Sequence Listing

SEQ ID NO: 1 (human IL-19)
MKLQCVSLWLLGTILILCSVDNHGLRRCLISTDMHHIEESFQEIKRAIQAKDTFPN
VTILSTLETLQIIKPLDVCCVTKNLLAFYVDRVFKDHQEPNPKILRKISSIANSFLY
MQKTLRQCQEQRQCHCRQEATNATRVIHDNYDQLEVHAAAIKSLGELDVFLAW
INKNHEVMFSA
SEQ ID NO: 2 (LC of murine derived IL-19 Ab1 (“M_1”))
DIVMTQSPSSLAMSVGQKVTMSCKSSQSLLSSYNQKNYLAWYQQKPGQSPKLLV
YFASTRPPGVPDRFIGSGSGSDFTLTISSVQAEDLADYFCQQHERLPITFGAGTKLE
LKRADAAPTVSIFPPSSEQLTSGGASVVCFLNNFYPKDINVKWKIDGSERQNGVL
NSWTDQDSKDSTYSMSSTLTLTKDEYERHNSYTCEATHKTSTSPIVKSFNRNEC
SEQ ID NO: 3 (LCDR1 of murine derived IL-19 Ab1)
KSSQSLLSSYNQKNYLA
SEQ ID NO: 4 (LCDR2 of murine derived IL-19 Ab1)
FASTRPP
SEQ ID NO: 5 (LCDR3 of murine derived IL-19 Ab1)
QQHERLPIT
SEQ ID NO: 6 (HC of murine derived IL-19 Ab1 (“M_1”))
EVQLQQSGPVLVKPGASVKMSCKASGYKFTDYFVGWVKQSHGKSLEWIGYVDP
ENGYTRCNQKFKGKATLTVDKSSSTAYMELNSLTSEDSAVYYCARYDYDWAWF
TNWGQGTLVTVSAAKTTPPSVYPLAPGSAAQTNSMVTLGCLVKGYFPEPVTVT
WNSGSLSSGVHTFPAVLQSDLYTLSSSVTVPSSTWPSETVTCNVAHPASSTKVDK
KIVPRDCGCKPCICTVPEVSSVFIFPPKPKDVLTITLTPKVTCVVVDISKDDPEVQF
SWFVDDVEVHTAQTQPREEQFNSTFRSVSELPIMHQDWLNGKEFKCRVNSAAFP
APIEKTISKTKGRPKAPQVYTIPPPKEQMAKDKVSLTCMITDFFPEDITVEWQWNG
QPAENYKNTQPIMDTDGSYFVYSKLNVQKSNWEAGNTFTCSVLHEGLHNHHTE
KSLSHSPGK
SEQ ID NO: 7 (HCDR1 of murine derived IL-19 Ab1)
GYKFTDYFVG
SEQ ID NO: 8 (HCDR2 of murine derived IL-19 Ab1)
YVDPENGYTRCNQKFKG
SEQ ID NO: 9 (HCDR3 of murine derived IL-19 Ab1)
YDYDWAWFTN
SEQ ID NO: 10 (LC of murine derived IL-19 Ab2 (“M_2”))
DIVLTQSPESLAVSLGQRATISCRASESVDNYGISFIHWYQQKPGQPPKLLIYRASN
LKSGIPARFSGRGSRTDFTLTINPVETDDVATYYCQQINKDPLTFGAGTKLELKRA
DAAPTVSIFPPSSEQLTSGGASVVCFLNNFYPKDINVKWKIDGSERQNGVLNSWT
DQDSKDSTYSMSSTLTLTKDEYERHNSYTCEATHKTSTSPIVKSFNRNEC
SEQ ID NO: 11 (LCDR1 of murine derived IL-19 Ab2)
RASESVDNYGISFIH
SEQ ID NO: 12 (LCDR2 of murine derived IL-19 Ab2)
RASNLKS
SEQ ID NO: 13 (LCDR3 of murine derived IL-19 Ab2)
QQINKDPLT
SEQ ID NO: 14 (HC of murine derived IL-19 Ab2 (“M_2”))
LVQLQQSDAELVKPGASLKISCKVSGYTFTDHALHWMKQRPEQGLEWIGYIYPR
DGSTKYNEKFKGKATLTADRSSSTAYMELNSLTSEDSAVYFCARGWDFAMDYW
GQGTSVTVSSAKTTPPSVYPLAPGSAAQTNSMVTLGCLVKGYFPEPVTVTWNSG
SLSSGVHTFPAVLQSDLYTLSSSVTVPSSTWPSETVTCNVAHPASSTKVDKKIVPR
DCGCKPCICTVPEVSSVFIFPPKPKDVLTITLTPKVTCVVVDISKDDPEVQFSWFVD
DVEVHTAQTQPREEQFNSTFRSVSELPIMHQDWLNGKEFKCRVNSAAFPAPIEKT
ISKTKGRPKAPQVYTIPPPKEQMAKDKVSLTCMITDFFPEDITVEWQWNGQPAEN
YKNTQPIMDTDGSYFVYSKLNVQKSNWEAGNTFTCSVLHEGLHNHHTEKSLSHS
PGK
SEQ ID NO: 15 (HCDR1 of murine derived IL-19 Ab2)
GYTFTDHALH
SEQ ID NO: 16 (HCDR2 of murine derived IL-19 Ab2)
YIYPRDGSTKYNEKFKG
SEQ ID NO: 17 (HCDR3 of murine derived IL-19 Ab2)
GWDFAMDY
SEQ ID NO: 18 (LC of murine derived IL-19 Ab3 (“M_3”))
DIKMTQSPSSMYASLGERVTITCKASQDINSYLSWFQQKPGKSPKTLIYRANRLV
DGVPSRFSGSGSGQDYSLTISSLEYEDMGIYFCLQYDDFPYTFGGGTKLEIKRADA
APTVSIFPPSSEQLTSGGASVVCFLNNFYPKDINVKWKIDGSERQNGVLNSWTDQ
DSKDSTYSMSSTLTLTKDEYERHNSYTCEATHKTSTSPIVKSFNRNEC
SEQ ID NO: 19 (LCDR1 of murine derived IL-19 Ab3)
KASQDINSYLS
SEQ ID NO: 20 (LCDR2 of murine derived IL-19 Ab3)
RANRLVD
SEQ ID NO: 21 (LCDR3 of murine derived IL-19 Ab3)
LQYDDFPYT
SEQ ID NO: 22 (HC of murine derived IL-19 Ab3 (“M_3”))
QIQLVQSGPELKKPGETVKISCKASGYTFTTYGMSWVKQAPGKGLKWMVWINT
YSGVPTYVDDFKGRFAFSLETSASTAYLQINNLKNEDTATYFCARRDEGFAYWG
QGTLVTVSAAKTTPPSVYPLAPGSAAQTNSMVTLGCLVKGYFPEPVTVTWNSGS
LSSGVHTFPAVLQSDLYTLSSSVTVPSSTWPSETVTCNVAHPASSTKVDKKIVPRD
CGCKPCICTVPEVSSVFIFPPKPKDVLTITLTPKVTCVVVDISKDDPEVQFSWFVDD
VEVHTAQTQPREEQFNSTFRSVSELPIMHQDWLNGKEFKCRVNSAAFPAPIEKTIS
KTKGRPKAPQVYTIPPPKEQMAKDKVSLTCMITDFFPEDITVEWQWNGQPAENY
KNTQPIMDTDGSYFVYSKLNVQKSNWEAGNTFTCSVLHEGLHNHHTEKSLSHSP
GK
SEQ ID NO: 23 (HCDR1 of murine derived IL-19 Ab3)
GYTFTTYGMS
SEQ ID NO: 24 (HCDR2 of murine derived IL-19 Ab3)
WINTYSGVPTYVDDFKG
SEQ ID NO: 25 (HCDR3 of murine derived IL-19 Ab3)
RDEGFAY
SEQ ID NO: 26 (LC of murine derived IL-19 Ab4 (“M_4”))
DIVMTQSPSSLAMSVGQKVTMSCKSSQSLLSSYNQKNYLAWYQQKPGQSPKLLV
YFASTRESGVPDRFIGSGSGSDFTLTISSVQAEDLADYFCQQHYSTPLTFGAGTKL
ELKRADAAPTVSIFPPSSEQLTSGGASVVCFLNNFYPKDINVKWKIDGSERQNGV
LNSWTDQDSKDSTYSMSSTLTLTKDEYERHNSYTCEATHKTSTSPIVKSFNRNEC
SEQ ID NO: 27 (LCDR1 of murine derived IL-19 Ab4)
KSSQSLLSSYNQKNYLA
SEQ ID NO: 28 (LCDR2 of murine derived IL-19 Ab4)
FASTRES
SEQ ID NO: 29 (LCDR3 of murine derived IL-19 Ab4)
QQHYSTPLT
SEQ ID NO: 30 (HC of murine derived IL-19 Ab4 (“M_4”))
EVQLQQSGPVLVKPGASVKMSCKASGYTFTDYYMNWVKQSHGKSLEWIGFINP
YNDDTRCNQKFKGKATLTVDKSSSTAYMELNSLTSEDSAVYYCARYDGYWAW
FAYWGQGTLVTVSAAKTTPPSVYPLAPGSAAQTNSMVTLGCLVKGYFPEPVTVT
WNSGSLSSGVHTFPAVLQSDLYTLSSSVTVPSSTWPSETVTCNVAHPASSTKVDK
KIVPRDCGCKPCICTVPEVSSVFIFPPKPKDVLTITLTPKVTCVVVDISKDDPEVQF
SWFVDDVEVHTAQTQPREEQFNSTFRSVSELPIMHQDWLNGKEFKCRVNSAAFP
APIEKTISKTKGRPKAPQVYTIPPPKEQMAKDKVSLTCMITDFFPEDITVEWQWNG
QPAENYKNTQPIMDTDGSYFVYSKLNVQKSNWEAGNTFTCSVLHEGLHNHHTE
KSLSHSPGK
SEQ ID NO: 31 (HCDR1 of murine derived IL-19 Ab4)
GYTFTDYYMN
SEQ ID NO: 32 (HCDR2 of murine derived IL-19 Ab4)
FINPYNDDTRCNQKFKG
SEQ ID NO: 33 (HCDR3 of murine derived IL-19 Ab4)
YDGYWAWFAY
SEQ ID NO: 34 (LCVR of rabbit derived IL-19 Ab1 (“R_1”))
AIDMTQTPASVSEPVGGTVTIKCQASQSISSYLAWYQQKPGQPPKLLIYSASTLAS
GVPSRFRGSGSGREFTLTISDLE€ADAATYYCQCHYHGSSYWDNSFGGGTEVVV
K
SEQ ID NO: 35 (LCDR1 of rabbit derived IL-19 Ab1)
QASQSISSYLA
SEQ ID NO: 36 (LCDR2 of rabbit derived IL-19 Ab1)
SASTLAS
SEQ ID NO: 37 (LCDR3 of rabbit derived IL-19 Ab1)
QCHYHGSSYWDNS
SEQ ID NO: 38 (HCVR of rabbit derived IL-19 Ab1 (“R_1”))
QQLEESGGGLVQPEGSLTLTCTASGFSFSSSYWICWVRQAPGKGLEWLACIDTGV
SGDTYYANWAEGRFTISKTSSTTVTLQMTSLTAADTATYFCARDIFGSAIDNSLW
GPGTLVTVSS
SEQ ID NO: 39 (HCDR1 of rabbit derived IL-19 Ab1)
GFSFSSSYWIC
SEQ ID NO: 40 (HCDR2 of rabbit derived IL-19 Ab1)
CIDTGVSGDTYYANWAEG
SEQ ID NO: 41 (HCDR3 of rabbit derived IL-19 Ab1)
DIFGSAIDNSL
SEQ ID NO: 42 (LCVR of rabbit derived IL-19 Ab2 (“R_2”))
ADVVMTQTPASVEAAVGGTVTIKCQASESIGNALAWYQQKPGQPPKLLIYDASK
LASGVPSRFKGSGSGTQFTLTISDLECADAATYYCQCHYHGSSYWDNSFGGGTE
VVVK
SEQ ID NO: 43 (LCDR1 of rabbit derived IL-19 Ab2)
QASESIGNALA
SEQ ID NO: 44 (LCDR2 of rabbit derived IL-19 Ab2)
DASKLAS
SEQ ID NO: 45 (LCDR3 of rabbit derived IL-19 Ab2)
QCHYHGSSYWDNS
SEQ ID NO: 46 (HCVR of rabbit derived IL-19 Ab2 (“R_2”))
QQLEESGGGLVQPEGSLTLTCTASGFSFSSSYWICWVRQAPGKGLEWLACIDTGV
SGDTYYANWAKGRFTISKTSSTTVTIQMTSLTAADTATYFCARDIFGSAIDNSLW
GPGTLVTVSS
SEQ ID NO: 47 (HCDR1 of rabbit derived IL-19 Ab2)
GFSFSSSYWIC
SEQ ID NO: 48 (HCDR2 of rabbit derived IL-19 Ab2)
CIDTGVSGDTYYANWAKG
SEQ ID NO: 49 (HCDR3 of rabbit derived IL-19 Ab2)
DIFGSAIDNSL
SEQ ID NO: 50 (LCVR of rabbit derived IL-19 Ab4 (“R_3”))
ADVVMTQTPSSVEAAVGGTVTIKCQASQSIGSSIAWYQQKPGQRPKLLIYGASTL
ASGVPSRFKGSGSGTEFTLTISDLECADAATYYCQCHYHGSSYWDNSFGGGTEV
VVK
SEQ ID NO: 51 (LCDR1 of rabbit derived IL-19 Ab3)
QASQSIGSSLA
SEQ ID NO: 52 (LCDR2 of rabbit derived IL-19 Ab3)
GASTLAS
SEQ ID NO: 53 (LCDR3 of rabbit derived IL-19 Ab3)
QCHYHGSSYWDNS
SEQ ID NO: 54 (HCVR of rabbit derived IL-19 Ab4 (“R_3”))
QCQSLEESGGGLVEPEGSLTLTCKASGFSFSSSYWICWVRQAPGKGLEWIACTDT
FSGDTYYASWAKGRFTISKTSSTTVTLRMTSLTDADTATYFCARDIFGTAVHISL
WGPGTLVTVSS
SEQ ID NO: 55 (HCDR1 of rabbit derived IL-19 Ab3)
GFSFSSSYWIC
SEQ ID NO: 56 (HCDR2 of rabbit derived IL-19 Ab3)
CTDTFSGDTYYASWAKG
SEQ ID NO: 57 (HCDR3 of rabbit derived IL-19 Ab3)
DIFGTAVHISL
SEQ ID NO: 58 (LCVR of rabbit derived IL-19 Ab5 (“R_4”))
ADVVMTQTPSSVSEPVGGTVTIKCQASQSIYSYLSWYQQKPGQPPKLLIYEASKL
ASGVPSRFKGSGSGTQFTLTISGVECADAATYYCQQGYSSSNVDNTFGGGTEVV
VT
SEQ ID NO: 59 (LCDR1 of rabbit derived IL-19 Ab4)
QASQSIYSYLS
SEQ ID NO: 60 (LCDR2 of rabbit derived IL-19 Ab4)
EASKLAS
SEQ ID NO: 61 (LCDR3 of rabbit derived IL-19 Ab4)
QQGYSSSNVDNT
SEQ ID NO: 62 (HCVR of rabbit derived IL-19 Ab5 (“R_4”))
QCQSVEESGGRLVTPGTPLTLTCTVSGIDLSIYAMGWVRQAPGKGLEYIGIIDTTG
TAYYARWAKGRFTISKTSATVALKITSPTTEDTATYFCARDSGSLYYGSYALWGP
GTLVTVSS
SEQ ID NO: 63 (HCDR1 of rabbit derived IL-19 Ab4)
GIDLSIYAMG
SEQ ID NO: 64 (HCDR2 of rabbit derived IL-19 Ab4)
IIDTTGTAYYARWAKG
SEQ ID NO: 65 (HCDR3 of rabbit derived IL-19 Ab4)
DSGSLYYGSYAL
SEQ ID NO: 66 (LCVR of phage derived IL-19 Ab1 (“P_1”))
EIVMTQSPGTLSLSPGERATLSCRASQSISSGYLAWYQQKPGQAPRLLIYGASSRA
TGIPDRFSGSGSGTDFTLTISRLEPEDFATYYCLQFKSFPLTFGQGTKLEIKR
SEQ ID NO: 67 (LCDR1 of phage derived IL-19 Ab1)
RASQSISSGYLA
SEQ ID NO: 68 (LCDR2 of phage derived IL-19 Ab1)
GASSRAT
SEQ ID NO: 69 (LCDR3 of phage derived IL-19 Ab1)
LQFKSFPLT
SEQ ID NO: 70 (HCVR of phage derived IL-19 Ab1 (“P_1”))
QVQLVQSGAEVKKPGSSVKVSCKASGGTFSSYAISWVRQAPGQGLEWMGGIIPIF
GTANYAQKFQGRVTITADESTSTAYMELSSLRSEDTAVYYCARDGGDYGDMGY
WGQGTLVTVSS
SEQ ID NO: 71 (HCDR1 of phage derived IL-19 Ab1)
GGTFSSYAIS
SEQ ID NO: 72 (HCDR2 of phage derived IL-19 Ab1)
GIIPIFGTANYAQKFQG
SEQ ID NO: 73 (HCDR3 of phage derived IL-19 Ab1)
DGGDYGDMGY
SEQ ID NO: 74 (LCVR of phage derived IL-19 Ab2 (“P_2”))
EIVMTQSPDSLAVSLGERATINCKSSQSVLYRSNSKSYLAWYQQKPGQPPKLLIY
WASTRESGVPDRFSGSGSGTDFTLTISSLQAEDVAVYYCQQYYSTPGSFGPGTKV
EIKR
SEQ ID NO: 75 (LCDR1 of phage derived IL-19 Ab2)
KSSQSVLYRSNSKSYLA
SEQ ID NO: 76 (LCDR2 of phage derived IL-19 Ab2)
WASTRES
SEQ ID NO: 77 (LCDR3 of phage derived IL-19 Ab2)
QQYYSTPGS
SEQ ID NO: 78 (HCVR of phage derived IL-19 Ab2 (“P_2”))
EVQLLESGGGLVQPGGSLRLSCAASGFTFSSYAMSWVRQAPGKGLEWVSAISQS
GGSTYYADSVKGRFTISRDNSKNTLYLQMNSLRAEDTAVYYCARHPERGSRRGP
YYYYMDVWGKGTTVTVSS
SEQ ID NO: 79 (HCDR1 of phage derived IL-19 Ab2)
GFTFSSYAMS
SEQ ID NO: 80 (HCDR2 of phage derived IL-19 Ab2)
AISQSGGSTYYADSVKG
SEQ ID NO: 81 (HCDR3 of phage derived IL-19 Ab2)
HPERGSRRGPYYYYMDV
SEQ ID NO: 82 (LCVR of phage derived IL-19 Ab3 (“P_3”))
DIQLTQSPSSLSASLGDRVTITCRASQGISSWLAWYQQKPGKAPKLLIYAASTLQS
GVPSRFSGSGSGTDFTLTISSLQPEDVATYYCQQYYDYSPWAFGQGTKLEIKR
SEQ ID NO: 83 (LCDR1 of phage derived IL-19 Ab3)
RASQGISSWLA
SEQ ID NO: 84 (LCDR2 of phage derived IL-19 Ab3)
AASTLQS
SEQ ID NO: 85 (LCDR3 of phage derived IL-19 Ab3)
QQYYDYSPWA
SEQ ID NO: 86 (HCVR of phage derived IL-19 Ab3 (“P_3”))
EVQLLESGGGLVQPGGSLRLSCAASGFTFSSYAMSWVRQAPGKGLEWVSAISGS
GGSTYYADSVKGRFTISRDNSKNTLYLQMNSLRAEDTAVYYCARGLSLLYYDLS
ENYFDYWGQGTTVTVSS
SEQ ID NO: 87 (HCDR1 of phage derived IL-19 Ab3)
GFTFSSYAMS
SEQ ID NO: 88 (HCDR2 of phage derived IL-19 Ab3)
AISGSGGSTYYADSVKG
SEQ ID NO: 89 (HCDR3 of phage derived IL-19 Ab3)
GLSLLYYDLSENYFDY
SEQ ID NO: 90 (LCDR1 of rabbit derived IL-19 Ab5)
QASQSIYSYLS
SEQ ID NO: 91 (LCDR2 of rabbit derived IL-19 Ab5)
EASKLAS
SEQ ID NO: 92 (LCDR3 of rabbit derived IL-19 Ab5)
QQGYSSSNVDNT
SEQ ID NO: 93 (HCDR1 of rabbit derived IL-19 Ab5)
GIDLSIYAMG
SEQ ID NO: 94 (HCDR2 of rabbit derived IL-19 Ab5)
IIDTTGTAYYARWAKG
SEQ ID NO: 95 (HCDR3 of rabbit derived IL-19 Ab5)
DSGSLYYGSYAL
SEQ ID NO: 96 (LCDR1 of rabbit derived IL-19 Ab6)
QASESIYSSLA
SEQ ID NO: 97 (LCDR2 of rabbit derived IL-19 Ab6)
GASTLAS
SEQ ID NO: 98 (LCDR3 of rabbit derived IL-19 Ab6)
QCHYHGSSYWDNS
SEQ ID NO: 99 (HCDR1 of rabbit derived IL-19 Ab6)
GFSFSSSYWIC
SEQ ID NO: 100 (HCDR2 of rabbit derived IL-19 Ab6)
CTDTFSGDTYYASWAKG
SEQ ID NO: 101 (HCDR3 of rabbit derived IL-19 Ab6)
DIFGTAVHISL
SEQ ID NO: 102 (LCDR1 of rabbit derived IL-19 Ab7)
QASESIYSSLA
SEQ ID NO: 103 (LCDR2 of rabbit derived IL-19 Ab7)
GASTLAS
SEQ ID NO: 104 (LCDR3 of rabbit derived IL-19 Ab7)
QCHYHGSSYWDNS
SEQ ID NO: 105 (HCDR1 of rabbit derived IL-19 Ab7)
GFSFSSSYWIC
SEQ ID NO: 106 (HCDR2 of rabbit derived IL-19 Ab7)
CTDTFSGDTYYASWAKG
SEQ ID NO: 107 (HCDR3 of rabbit derived IL-19 Ab7)
DIFGTAVHISL
SEQ ID NO: 108 (LCDR1 of rabbit derived IL-19 Ab8)
QASQSIGSSLA
SEQ ID NO: 109 (LCDR2 of rabbit derived IL-19 Ab8)
GASTLAS
SEQ ID NO: 110 (LCDR3 of rabbit derived IL-19 Ab8)
QCHYHGSSYWDNS
SEQ ID NO: 111 (HCDR1 of rabbit derived IL-19 Ab8)
GFSFSSSYWIC
SEQ ID NO: 112 (HCDR2 of rabbit derived IL-19 Ab8)
CTDTFSGDTYYASWAKG
SEQ ID NO: 113 (HCDR3 of rabbit derived IL-19 Ab8)
DIFGTAVHISL
SEQ ID NO: 114 (LCDR1 of rabbit derived IL-19 Ab9)
QASESIYSSLA
SEQ ID NO: 115 (LCDR2 of rabbit derived IL-19 Ab9)
GASTLAS
SEQ ID NO: 116 (LCDR3 of rabbit derived IL-19 Ab9)
QCHYHGSSYWDNS
SEQ ID NO: 117 (HCDR1 of rabbit derived IL-19 Ab9)
GFSFSSSYWIC
SEQ ID NO: 118 (HCDR2 of rabbit derived IL-19 Ab9)
CTDTFSGDTYYASWAKG
SEQ ID NO: 119 (HCDR3 of rabbit derived IL-19 Ab9)
DIFGTAVHISL
SEQ ID NO: 120 (LCDR1 of rabbit derived IL-19 Ab10)
QASQSISSYLA
SEQ ID NO: 121 (LCDR2 of rabbit derived IL-19 Ab10)
GASTLAS
SEQ ID NO: 122 (LCDR3 of rabbit derived IL-19 Ab10)
QCHYHGSSYWDNS
SEQ ID NO: 123 (HCDR1 of rabbit derived IL-19 Ab10)
GFSFSSSYWIC
SEQ ID NO: 124 (HCDR2 of rabbit derived IL-19 Ab10)
CTDTFSGDTYYASWAKG
SEQ ID NO: 125 (HCDR3 of rabbit derived IL-19 Ab10)
DIFGTAVHISL
SEQ ID NO: 126 (LCDR1 of rabbit derived IL-19 Ab11)
QASQSISSYLA
SEQ ID NO: 127 (LCDR2 of rabbit derived IL-19 Ab11)
GASTLAS
SEQ ID NO: 128 (LCDR3 of rabbit derived IL-19 Ab11)
QSAVYSSSSGYGVP
SEQ ID NO: 129 (HCDR1 of rabbit derived IL-19 Ab11)
GFSLSSYDMS
SEQ ID NO: 130 (HCDR2 of rabbit derived IL-19 Ab11)
IIDSIGSIWYANWAKG
SEQ ID NO: 131 (HCDR3 of rabbit derived IL-19 Ab11)
ESGPINTDYDL
SEQ ID NO: 132 (LCDR1 of rabbit derived IL-19 Ab12)
QTSESFYSNNILS
SEQ ID NO: 133 (LCDR2 of rabbit derived IL-19 Ab12)
EASKLAS
SEQ ID NO: 134 (LCDR3 of rabbit derived IL-19 Ab12)
QSAIYDGSYIVT
SEQ ID NO: 135 (HCDR1 of rabbit derived IL-19 Ab12)
GFSLSSYDMS
SEQ ID NO: 136 (HCDR2 of rabbit derived IL-19 Ab12)
IIDSIGSIWYANWAKG
SEQ ID NO: 137 (HCDR3 of rabbit derived IL-19 Ab12)
ESGPINTDYDL
SEQ ID NO: 138 (LCDR1 of rabbit derived IL-19 Ab13)
QASQSISSYLA
SEQ ID NO: 139 (LCDR2 of rabbit derived IL-19 Ab13)
GASTLAS
SEQ ID NO: 140 (LCDR3 of rabbit derived IL-19 Ab13)
QSAVYSSSSGYGVP
SEQ ID NO: 141 (HCDR1 of rabbit derived IL-19 Ab13)
GFSLSSYDMS
SEQ ID NO: 142 (HCDR2 of rabbit derived IL-19 Ab13)
IIDSIGSIWYANWAKG
SEQ ID NO: 143 (HCDR3 of rabbit derived IL-19 Ab13)
ESGPINTDYDL
SEQ ID NO: 144 (LCDR1 of rabbit derived IL-19 Ab14)
QASQSISSYLA
SEQ ID NO: 145 (LCDR2 of rabbit derived IL-19 Ab14)
GASTLAS
SEQ ID NO: 146 (LCDR3 of rabbit derived IL-19 Ab14)
QSAVYSSSSGYGVP
SEQ ID NO: 147 (HCDR1 of rabbit derived IL-19 Ab14)
GFSLSSYDMS
SEQ ID NO: 148 (HCDR2 of rabbit derived IL-19 Ab14)
IIDSIGSIWYANWAKG
SEQ ID NO: 149 (HCDR3 of rabbit derived IL-19 Ab14)
ESGPINTDYDL
SEQ ID NO: 150 (LCDR1 of rabbit derived IL-19 Ab15)
QASESISSYLA
SEQ ID NO: 151 (LCDR2 of rabbit derived IL-19 Ab15)
GASTLAS
SEQ ID NO: 152 (LCDR3 of rabbit derived IL-19 Ab15)
QGYFGDYIYG
SEQ ID NO: 153 (HCDR1 of rabbit derived IL-19 Ab15)
GFSLTSYAMS
SEQ ID NO: 154 (HCDR2 of rabbit derived IL-19 Ab15)
IIGSPGTTGYATWAKG
SEQ ID NO: 155 (HCDR3 of rabbit derived IL-19 Ab15)
GWFYYGMAL
SEQ ID NO: 156 (LCDR1 of rabbit derived IL-19 Ab16)
PXQEHLTP
SEQ ID NO: 157 (LCDR2 of rabbit derived IL-19 Ab16)
GASTLAX
SEQ ID NO: 158 (LCDR3 of rabbit derived IL-19 Ab16)
QGYFGDYIYG
SEQ ID NO: 159 (HCDR1 of rabbit derived IL-19 Ab16)
GFSLTSYAMS
SEQ ID NO: 160 (HCDR2 of rabbit derived IL-19 Ab16)
IIGSPGTTGYATWAKG
SEQ ID NO: 161 (HCDR3 of rabbit derived IL-19 Ab16)
GWFYYGMAL
SEQ ID NO: 162 (LCDR1 of rabbit derived IL-19 Ab17)
QASESISSYLA
SEQ ID NO: 163 (LCDR2 of rabbit derived IL-19 Ab17)
GASTLAS
SEQ ID NO: 164 (LCDR3 of rabbit derived IL-19 Ab17)
QGYFGDYIYG
SEQ ID NO: 165 (HCDR1 of rabbit derived IL-19 Ab17)
GFSLTSYAMS
SEQ ID NO: 166 (HCDR2 of rabbit derived IL-19 Ab17)
IIGSPGTTGYATWAKG
SEQ ID NO: 167 (HCDR3 of rabbit derived IL-19 Ab17)
GWFYYGMAL
SEQ ID NO: 168 (LCDR1 of rabbit derived IL-19 Ab18)
QASESISSYLA
SEQ ID NO: 169 (LCDR2 of rabbit derived IL-19 Ab18)
GASTLAS
SEQ ID NO: 170 (LCDR3 of rabbit derived IL-19 Ab18)
QGYFGDYIYG
SEQ ID NO: 171 (HCDR1 of rabbit derived IL-19 Ab18)
GFSLTSYAMS
SEQ ID NO: 172 (HCDR2 of rabbit derived IL-19 Ab18)
IIGSPGTTGYATWAKG
SEQ ID NO: 173 (HCDR3 of rabbit derived IL-19 Ab18)
GWFYYGMAL
SEQ ID NO: 174 (LCDR1 of rabbit derived IL-19 Ab19)
QASESISSYLA
SEQ ID NO: 175 (LCDR2 of rabbit derived IL-19 Ab19)
GASTLAS
SEQ ID NO: 176 (LCDR3 of rabbit derived IL-19 Ab19)
QGYFGDYIYG
SEQ ID NO: 177 (HCDR1 of rabbit derived IL-19 Ab19)
GFSLTSYAMS
SEQ ID NO: 178 (HCDR2 of rabbit derived IL-19 Ab19)
IIGSPGTTGYATWAKG
SEQ ID NO: 179 (HCDR3 of rabbit derived IL-19 Ab19)
GWFYYGMAL
SEQ ID NO: 180 (LCDR1 of rabbit derived IL-19 Ab20)
QASESISSYLA
SEQ ID NO: 181 (LCDR2 of rabbit derived IL-19 Ab20)
GASTLAS
SEQ ID NO: 182 (LCDR3 of rabbit derived IL-19 Ab20)
QGYFGDYIYG
SEQ ID NO: 183 (HCDR1 of rabbit derived IL-19 Ab20)
GFSLTSYAMS
SEQ ID NO: 184 (HCDR2 of rabbit derived IL-19 Ab20)
IIGSPGTTGYATWAKG
SEQ ID NO: 185 (HCDR3 of rabbit derived IL-19 Ab20)
GWFYYGMAL
SEQ ID NO: 186 (LCDR1 of rabbit derived IL-19 Ab21)
QASQSISNYLS
SEQ ID NO: 187 (LCDR2 of rabbit derived IL-19 Ab21)
WASNLAS
SEQ ID NO: 188 (LCDR3 of rabbit derived IL-19 Ab21)
LGEFSCSSADCFA
SEQ ID NO: 189 (HCDR1 of rabbit derived IL-19 Ab21)
GFDLSNYAMT
SEQ ID NO: 190 (HCDR2 of rabbit derived IL-19 Ab21)
AIHGSGVTDCASWTRG
SEQ ID NO: 191 (HCDR3 of rabbit derived IL-19 Ab21)
ESAGINTDYDL
SEQ ID NO: 192 (LCDR1 of rabbit derived IL-19 Ab22)
QASQSISNYLS
SEQ ID NO: 193 (LCDR2 of rabbit derived IL-19 Ab22)
WASNLAS
SEQ ID NO: 194 (LCDR3 of rabbit derived IL-19 Ab22)
LGEFSCSSADCFA
SEQ ID NO: 195 (HCDR1 of rabbit derived IL-19 Ab22)
GFDLSNYAMT
SEQ ID NO: 196 (HCDR2 of rabbit derived IL-19 Ab22)
AIHGSGVTDCASWTRG
SEQ ID NO: 197 (HCDR3 of rabbit derived IL-19 Ab22)
ESAGINTDYDL
SEQ ID NO: 198 (LCDR1 of rabbit derived IL-19 Ab23)
QASKSIGSYLS
SEQ ID NO: 199 (LCDR2 of rabbit derived IL-19 Ab23)
RASTLAS
SEQ ID NO: 200 (LCDR3 of rabbit derived IL-19 Ab23)
QAYYGDYIYD
SEQ ID NO: 201 (HCDR1 of rabbit derived IL-19 Ab23)
GFSLSSYPMS
SEQ ID NO: 202 (HCDR2 of rabbit derived IL-19 Ab23)
IIGSTGSTGYANWAKG
SEQ ID NO: 203 (HCDR3 of rabbit derived IL-19 Ab23)
GWFYYGLDL
SEQ ID NO: 204 (LCDR1 of rabbit derived IL-19 Ab24)
QASQSIGSYLS
SEQ ID NO: 205 (LCDR2 of rabbit derived IL-19 Ab24)
RASTLAS
SEQ ID NO: 206 (LCDR3 of rabbit derived IL-19 Ab24)
QAYYGDYIYD
SEQ ID NO: 207 (HCDR1 of rabbit derived IL-19 Ab24)
GFSLSSYPMS
SEQ ID NO: 208 (HCDR2 of rabbit derived IL-19 Ab24)
IIGSTGSTGYANWAKG
SEQ ID NO: 209 (HCDR3 of rabbit derived IL-19 Ab24)
GWFYYGLDL
SEQ ID NO: 210 (LCDR1 of rabbit derived IL-19 Ab25)
QASQSIGSYLA
SEQ ID NO: 211 (LCDR2 of rabbit derived IL-19 Ab25)
DASDLAS
SEQ ID NO: 212 (LCDR3 of rabbit derived IL-19 Ab25)
QSAVYDSSYVVT
SEQ ID NO: 213 (HCDR1 of rabbit derived IL-19 Ab25)
GFSLSSYDMS
SEQ ID NO: 214 (HCDR2 of rabbit derived IL-19 Ab25)
ISDSLGTTWYANWAKG
SEQ ID NO: 215 (HCDR3 of rabbit derived IL-19 Ab25)
ESGDIRIDYDL
SEQ ID NO: 216 (LCDR1 of rabbit derived IL-19 Ab26)
QASQSIGSYLA
SEQ ID NO: 217 (LCDR2 of rabbit derived IL-19 Ab26)
DASDLAS
SEQ ID NO: 218 (LCDR3 of rabbit derived IL-19 Ab26)
QSAVYDSSYVVT
SEQ ID NO: 219 (HCDR1 of rabbit derived IL-19 Ab26)
GFSLSSYDMS
SEQ ID NO: 220 (HCDR2 of rabbit derived IL-19 Ab26)
ISDSLGTTWYANWAKG
SEQ ID NO: 221 (HCDR3 of rabbit derived IL-19 Ab26)
ESGDIRIDYDL
SEQ ID NO: 222 (LCDR1 of rabbit derived IL-19 Ab27)
QASQSIGSYLA
SEQ ID NO: 223 (LCDR2 of rabbit derived IL-19 Ab27)
DASDLAS
SEQ ID NO: 224 (LCDR3 of rabbit derived IL-19 Ab27)
QSAVYDSSYVVT
SEQ ID NO: 225 (HCDR1 of rabbit derived IL-19 Ab27)
GFSLSSYDMS
SEQ ID NO: 226 (HCDR2 of rabbit derived IL-19 Ab27)
ISDSLGTTWYANWAKG
SEQ ID NO: 227 (HCDR3 of rabbit derived IL-19 Ab27)
ESGDIRIDYDL
SEQ ID NO: 228 (LCDR1 of rabbit derived IL-19 Ab28)
QASQSIGSYLA
SEQ ID NO: 229 (LCDR2 of rabbit derived IL-19 Ab28)
DASDLAS
SEQ ID NO: 230 (LCDR3 of rabbit derived IL-19 Ab28)
QSAVYDSSYVVT
SEQ ID NO: 231 (HCDR1 of rabbit derived IL-19 Ab28)
GFSLSSYDMS
SEQ ID NO: 232 (HCDR2 of rabbit derived IL-19 Ab28)
ISDSLGTTWYANWAKG
SEQ ID NO: 233 (HCDR3 of rabbit derived IL-19 Ab28)
ESGDIRIDYDL
SEQ ID NO: 234 (LCDR1 of rabbit derived IL-19 Ab29)
QASQSLNINYLS
SEQ ID NO: 235 (LCDR2 of rabbit derived IL-19 Ab29)
SASTLAS
SEQ ID NO: 236 (LCDR3 of rabbit derived IL-19 Ab29)
QQSVNYGRVDNI
SEQ ID NO: 237 (HCDR1 of rabbit derived IL-19 Ab29)
GFTISNYHMS
SEQ ID NO: 238 (HCDR2 of rabbit derived IL-19 Ab29)
RIVSYGGTYYANWAKG
SEQ ID NO: 239 (HCDR3 of rabbit derived IL-19 Ab29)
DSSGSGFSFAL
SEQ ID NO: 240 (LCDR1 of rabbit derived IL-19 Ab30)
QASQSITTSYLS
SEQ ID NO: 241 (LCDR2 of rabbit derived IL-19 Ab30)
KASTLAS
SEQ ID NO: 242 (LCDR3 of rabbit derived IL-19 Ab30)
QQSHNYGRVDNI
SEQ ID NO: 243 (HCDR1 of rabbit derived IL-19 Ab30)
GFTISNYHMS
SEQ ID NO: 244 (HCDR2 of rabbit derived IL-19 Ab30)
RIVSYGGTYYANWAKG
SEQ ID NO: 245 (HCDR3 of rabbit derived IL-19 Ab30)
DSSGSGFSFAL
SEQ ID NO: 246 (LCDR1 of rabbit derived IL-19 Ab31)
QASQSITTSYLS
SEQ ID NO: 247 (LCDR2 of rabbit derived IL-19 Ab31)
KASTLAS
SEQ ID NO: 248 (LCDR3 of rabbit derived IL-19 Ab31)
QQSHNYGRVDNI
SEQ ID NO: 249 (HCDR1 of rabbit derived IL-19 Ab31)
GFTISNYHMS
SEQ ID NO: 250 (HCDR2 of rabbit derived IL-19 Ab31)
RIVSYGGTYYANWAKG
SEQ ID NO: 251 (HCDR3 of rabbit derived IL-19 Ab31)
DSSGSGFSFAL
SEQ ID NO: 252 (LCDR1 of rabbit derived IL-19 Ab32)
QASQSITTSYLS
SEQ ID NO: 253 (LCDR2 of rabbit derived IL-19 Ab32)
KASTLAS
SEQ ID NO: 254 (LCDR3 of rabbit derived IL-19 Ab32)
QQSHNYGRVDNI
SEQ ID NO: 255 (HCDR1 of rabbit derived IL-19 Ab32)
GFTISNYHMS
SEQ ID NO: 256 (HCDR2 of rabbit derived IL-19 Ab32)
RIVSYGGTYYANWAKG
SEQ ID NO: 257 (HCDR3 of rabbit derived IL-19 Ab32)
DSSGSGFSFAL
SEQ ID NO: 258 (LCDR1 of rabbit derived IL-19 Ab33)
QASQSITTSYLS
SEQ ID NO: 259 (LCDR2 of rabbit derived IL-19 Ab33)
KASTLAS
SEQ ID NO: 260 (LCDR3 of rabbit derived IL-19 Ab33)
QQSHNYGRVDNI
SEQ ID NO: 261 (HCDR1 of rabbit derived IL-19 Ab33)
GFTISNYHMS
SEQ ID NO: 262 (HCDR2 of rabbit derived IL-19 Ab33)
RIVSYGGTYYANWAKG
SEQ ID NO: 263 (HCDR3 of rabbit derived IL-19 Ab33)
DSSGSGFSFAL
SEQ ID NO: 264 (LCDR1 of rabbit derived IL-19 Ab34)
QASQNIYSNLA
SEQ ID NO: 265 (LCDR2 of rabbit derived IL-19 Ab34)
KASTLAS
SEQ ID NO: 266 (LCDR3 of rabbit derived IL-19 Ab34)
QAYVGNYIYT
SEQ ID NO: 267 (HCDR1 of rabbit derived IL-19 Ab34)
GFSLSSYPMS
SEQ ID NO: 268 (HCDR2 of rabbit derived IL-19 Ab34)
IIGSSGSIGYASWAKG
SEQ ID NO: 269 (HCDR3 of rabbit derived IL-19 Ab34)
GWFYYGMDL
SEQ ID NO: 270 (LCDR1 of rabbit derived IL-19 Ab35)
QASQSIGSNLA
SEQ ID NO: 271 (LCDR2 of rabbit derived IL-19 Ab35)
RASTLAS
SEQ ID NO: 272 (LCDR3 of rabbit derived IL-19 Ab35)
QGYYGDGIYG
SEQ ID NO: 273 (HCDR1 of rabbit derived IL-19 Ab35)
GFSLSSYPMS
SEQ ID NO: 274 (HCDR2 of rabbit derived IL-19 Ab35)
LIGSSGTIYFATWAKG
SEQ ID NO: 275 (HCDR3 of rabbit derived IL-19 Ab35)
GWFYYGMDL
SEQ ID NO: 276 (LCDR1 of rabbit derived IL-19 Ab36)
QASQSISSWLS
SEQ ID NO: 277 (LCDR2 of rabbit derived IL-19 Ab36)
GASTLAS
SEQ ID NO: 278 (LCDR3 of rabbit derived IL-19 Ab36)
QSYYYISSSSYT
SEQ ID NO: 279 (HCDR1 of rabbit derived IL-19 Ab36)
GFSLSSYPMS
SEQ ID NO: 280 (HCDR2 of rabbit derived IL-19 Ab36)
LIGSSGTIYFATWAKG
SEQ ID NO: 281 (HCDR3 of rabbit derived IL-19 Ab36)
GWFYYGMDL
SEQ ID NO: 282 (LCDR1 of rabbit derived IL-19 Ab37)
QASQSISSYLA
SEQ ID NO: 283 (LCDR2 of rabbit derived IL-19 Ab37)
SASTLAS
SEQ ID NO: 284 (LCDR3 of rabbit derived IL-19 Ab37)
QCHYHGSSYWDNS
SEQ ID NO: 285 (HCDR1 of rabbit derived IL-19 Ab37)
GFSFSSSYWIC
SEQ ID NO: 286 (HCDR2 of rabbit derived IL-19 Ab37)
CIDTGVSGDTYYANWAEG
SEQ ID NO: 287 (HCDR3 of rabbit derived IL-19 Ab37)
DIFGSAIDNSL
SEQ ID NO: 288 (LCDR1 of rabbit derived IL-19 Ab38)
QASQNIYSNLA
SEQ ID NO: 289 (LCDR2 of rabbit derived IL-19 Ab38)
GASNLES
SEQ ID NO: 290 (LCDR3 of rabbit derived IL-19 Ab38)
QGGYSGGIYA
SEQ ID NO: 291 (HCDR1 of rabbit derived IL-19 Ab38)
GFSLTRHAMS
SEQ ID NO: 292 (HCDR2 of rabbit derived IL-19 Ab38)
AIHGSGVTDYASWAKG
SEQ ID NO: 293 (HCDR3 of rabbit derived IL-19 Ab38)
GSGL
SEQ ID NO: 294 (LCVR of rabbit derived IL-19 Ab39 (“R_39”))
DIVMTQTPASVEAAVGGTVTIKCQASQSIYRYLSWYQQKPGQRPKLLIYGASNLA
SGVSSRFKGSGSGTEFTLTISDLECADAATYYCQSYYYSSSSGYGNVFGGGTEVV
VK
SEQ ID NO: 295 (LCDR1 of rabbit derived IL-19 Ab39)
QASQSIYRYLS
SEQ ID NO: 296 (LCDR2 of rabbit derived IL-19 Ab39)
GASNLAS
SEQ ID NO: 297 (LCDR3 of rabbit derived IL-19 Ab39)
QSYYYSSSSGYGNV
SEQ ID NO: 298 (HCVR of rabbit derived IL-19 Ab39 (“R_39”))
QEQLKESGGGLVQPGGSLKLSCKASGFDLSSYGVSWVRQAPGKGLEWIGYIDPV
FGSTYYANWVNGRFTISSDNAQNTLYLQLNSLTAADTATYFCARGSGYVYYGYT
YDLWGPGTLVTVSS
SEQ ID NO: 299 (HCDR1 of rabbit derived IL-19 Ab39)
GFDLSSYGVS
SEQ ID NO: 300 (HCDR2 of rabbit derived IL-19 Ab39)
YIDPVFGSTYYANWVNG
SEQ ID NO: 301 (HCDR3 of rabbit derived IL-19 Ab39)
GSGYVYYGYTYDL
SEQ ID NO: 302 (LCVR of murine derived IL-19 Ab5 (“M_5”))
DIVMTQSPSSLAMSVGQKVTMSCKSSQSLLSSYNQKNYLAWYQRKPGQSPKLLV
YFASQRATGVPDRFIGSGSGSDFTLTISSVQAEDLADYFCQQHTRLPITFGAGTKL
ELK
SEQ ID NO: 303 (LCDR1 of murine derived IL-19 Ab5)
KSSQSLLSSYNQKNYLA
SEQ ID NO: 304 (LCDR2 of murine derived IL-19 Ab5)
FASQRAT
SEQ ID NO: 305 (LCDR3 of murine derived IL-19 Ab5)
QQHTRLPIT
SEQ ID NO: 306 (HCVR of murine derived IL-19 Ab5 (“M_5”))
EVQLQQSGPVLVKPGASVKMSCKASGYPFNDYFLGWVKQSHGKSLEWIGSINPD
NDYTMSNQKFKGKATLTVDKSSSTAYMELNSLTSEDSAVYYCARYDYDWAWF
AHWGQGTLVTVSA
SEQ ID NO: 307 (HCDR1 of murine derived IL-19 Ab5)
GYPFNDYFLG
SEQ ID NO: 308 (HCDR2 of murine derived IL-19 Ab5)
SINPDNDYTMSNQKFKG
SEQ ID NO: 309 (HCDR3 of murine derived IL-19 Ab5)
YDYDWAWFAH
SEQ ID NO: 310 (LC of rabbit derived IL-19 Ab39 (“R_39”))
DIVMTQTPASVEAAVGGTVTIKCQASQSIYRYLSWYQQKPGQRPKLLIYGASNLA
SGVSSRFKGSGSGTEFTLTISDLECADAATYYCQSYYYSSSSGYGNVFGGGTEVV
VKGDPVAPTVLIFPPAADQVATGTVTIVCVANKYFPDVTVTWEVDGTTQTTGIEN
SKTPQNSADCTYNLSSTLTLTSTQYNSHKEYTCKVTQGTTSVVQSFNRGDC
SEQ ID NO: 311 (HC of rabbit derived IL-19 Ab39 (“R_39”))
QEQLKESGGGLVQPGGSLKLSCKASGFDLSSYGVSWVRQAPGKGLEWIGYIDPV
FGSTYYANWVNGRFTISSDNAQNTLYLQLNSLTAADTATYFCARGSGYVYYGYT
YDLWGPGTLVTVSSGQPKAPSVFPLAPCCGDTPSSTVTLGCLVKGYLPEPVTVT
WNSGTLTNGVRTFPSVRQSSGLYSLSSVVSVTSSSQPVTCNVAHPATNTKVDKTV
APSTCSKPTCPPPELLGGPSVFIFPPKPKDTLMISRTPEVTCVVVDVSQDDPEVQFT
WYINNEQVRTARPPLREQQFNSTIRVVSTLPIAHQDWLRGKEFKCKVHNKALPAP
IEKTISKARGQPLEPKVYTMGPPREELSSRSVSLTCMINGFYPSDISVEWEKNGKA
EDNYKTTPAVLDSDGSYFLYSKLSVPTSEWQRGDVFTCSVMHEALHNHYTQKSI
SRSPGK
SEQ ID NO: 312 (LC of murine derived IL-19 Ab5 (“M_5”))
DIVMTQSPSSLAMSVGQKVTMSCKSSQSLLSSYNQKNYLAWYQRKPGQSPKLLV
YFASQRATGVPDRFIGSGSGSDFTLTISSVQAEDLADYFCQQHTRLPITFGAGTKL
ELKRADAAPTVSIFPPSSEQLTSGGASVVCFLNNFYPKDINVKWKIDGSERQNGV
LNSWTDQDSKDSTYSMSSTLTLTKDEYERHNSYTCEATHKTSTSPIVKSFNRNEC
SEQ ID NO: 313 (HC of murine derived IL-19 Ab5 (“M_5”))
EVQLQQSGPVLVKPGASVKMSCKASGYPFNDYFLGWVKQSHGKSLEWIGSINPD
NDYTMSNQKFKGKATLTVDKSSSTAYMELNSLTSEDSAVYYCARYDYDWAWF
AHWGQGTLVTVSAAKTTPPSVYPLAPGSAAQTNSMVTLGCLVKGYFPEPVTVT
WNSGSLSSGVHTFPAVLQSDLYTLSSSVTVPSSTWPSETVTCNVAHPASSTKVDK
KIVPRDCGCKPCICTVPEVSSVFIFPPKPKDVLTITLTPKVTCVVVDISKDDPEVQF
SWFVDDVEVHTAQTQPREEQFNSTFRSVSELPIMHQDWLNGKEFKCRVNSAAFP
APIEKTISKTKGRPKAPQVYTIPPPKEQMAKDKVSLTCMITDFFPEDITVEWQWNG
QPAENYKNTQPIMDTDGSYFVYSKLNVQKSNWEAGNTFTCSVLHEGLHNHHTE
KSLSHSPGK

We claim:

1. An antibody which binds to human IL-19, said antibody comprising a light chain variable region (LCVR) and a heavy chain variable region (HCVR), wherein the LCVR comprises complementarity determining regions (CDRs) LCDR1, LCDR2, and LCDR3 and the HCVR comprises CDRs HCDR1, HCDR2 and HCDR3 selected from:
a. a grouping of LCDR1, LCDR2, LCDR3, HCDR1, HCDR2, and HCDR3 having at least 95% amino acid sequence homology to the amino acid sequence of a grouping of LCDR1, LCDR2, LCDR3, HCDR1, HCDR2, and HCDR3 provided in Table 1;
b. grouping of LCDR1, LCDR2, LCDR3, HCDR1, HCDR2, and HCDR3 having at least 95% amino acid sequence homology to the amino acid sequence of a grouping of LCDR1, LCDR2, LCDR3, HCDR1, HCDR2, and HCDR3 provided in Table 2; or
c. grouping of LCDR1, LCDR2, LCDR3, HCDR1, HCDR2, and HCDR3 having at least 95% amino acid sequence homology to the amino acid sequence of a grouping of LCDR1, LCDR2, LCDR3, HCDR1, HCDR2, and HCDR3 provided in Table 3.

2. The antibody of claim 1, wherein LCDR1, LCDR2, LCDR3, HCDR1, HCDR2 and HCDR3 are selected from:
a. a grouping of LCDR1, LCDR2, LCDR3, HCDR1, HCDR2, and HCDR3 provided in Table 1 and having the amino acid sequence of the grouping of LCDR1, LCDR2, LCDR3, HCDR1, HCDR2, and HCDR3 provided in Table 1;
b. a grouping of LCDR1, LCDR2, LCDR3, HCDR1, HCDR2, and HCDR3 provided in Table 2 and having the amino acid sequence of the grouping of LCDR1, LCDR2, LCDR3, HCDR1, HCDR2, and HCDR3 provided in Table 2; or
c. a grouping of LCDR1, LCDR2, LCDR3, HCDR1, HCDR2, and HCDR3 provided in Table 3 and having the amino acid sequence of the grouping of LCDR1, LCDR2, LCDR3, HCDR1, HCDR2, and HCDR3 provided in Table 3.

3. The antibody of claim 1, wherein the LCVR and the HCVR are selected from:
a. the LCVR having an amino acid sequence with at least 95% homology to the amino acid sequence of SEQ ID NO: 66 and the HCVR having an amino acid sequence with at least 95% homology to the amino acid sequence of SEQ ID NO: 70;
b. the LCVR having an amino acid sequence with at least 95% homology to the amino acid sequence of SEQ ID NO: 74 and the HCVR having an amino acid sequence with at least 95% homology to the amino acid sequence of SEQ ID NO: 78;
c. the LCVR having an amino acid sequence with at least 95% homology to the amino acid sequence of SEQ ID NO: 82 and the HCVR having an amino acid sequence with at least 95% homology to the amino acid sequence of SEQ ID NO: 86;
d. the LCVR having an amino acid sequence with at least 95% homology to the amino acid sequence of SEQ ID NO: 34 and the HCVR having an amino acid sequence with at least 95% homology to the amino acid sequence of SEQ ID NO: 38;
c. the LCVR having an amino acid sequence with at least 95% homology to the amino acid sequence of SEQ ID NO: 42 and the HCVR having an amino acid sequence with at least 95% homology to the amino acid sequence of SEQ ID NO: 46;
f. the LCVR having an amino acid sequence with at least 95% homology to the amino acid sequence of SEQ ID NO: 50 and the HCVR having an amino acid sequence with at least 95% homology to the amino acid sequence of SEQ ID NO: 54;
g. the LCVR having an amino acid sequence with at least 95% homology to the amino acid sequence of SEQ ID NO: 58 and the HCVR having an amino acid sequence with at least 95% homology to the amino acid sequence of SEQ ID NO: 62;
h. the LCVR having an amino acid sequence with at least 95% homology to the amino acid sequence of SEQ ID NO: 294 and the HCVR having an amino acid sequence with at least 95% homology to the amino acid sequence of SEQ ID NO: 298; and
i. the LCVR having an amino acid sequence with at least 95% homology to the amino acid sequence of SEQ ID NO: 302 and the HCVR having an amino acid sequence with at least 95% homology to the amino acid sequence of SEQ ID NO: 306.

4. The antibody of claim 2, wherein LCVR and HCVR are selected from:
a. the LCVR having the amino acid sequence of SEQ ID NO: 66 and the HCVR having the amino acid sequence of SEQ ID NO: 70;
b. the LCVR having the amino acid sequence of SEQ ID NO: 74 and the HCVR having the amino acid sequence of SEQ ID NO: 78;
c. the LCVR having the amino acid sequence of SEQ ID NO: 82 and the HCVR having the amino acid sequence of SEQ ID NO: 86;
d. the LCVR having the amino acid sequence of SEQ ID NO: 34 and the HCVR having the amino acid sequence of SEQ ID NO: 38;
e. the LCVR having the amino acid sequence of SEQ ID NO: 42 and the HCVR having the amino acid sequence of SEQ ID NO: 46;
f. the LCVR having the amino acid sequence of SEQ ID NO: 50 and the HCVR having the amino acid sequence of SEQ ID NO: 54;
g. the LCVR having the amino acid sequence of SEQ ID NO: 58 and the HCVR having the amino acid sequence of SEQ ID NO: 62;
h. the LCVR having the amino acid sequence of SEQ ID NO: 294 and the HCVR having the amino acid sequence of SEQ ID NO: 298; and
i. the LCVR having the amino acid sequence of SEQ ID NO: 302 and the HCVR having the amino acid sequence of SEQ ID NO: 306.

5. The antibody of claim 1 further comprising a IgG1 heavy chain.

6. The antibody of claim 1 further comprising a kappa light chain.

7. A method of treating an immune-mediated disease comprising administering to a patient in need thereof an effective amount of an antibody of claim 1.

8. The method of claim 7, wherein the immune-mediated disease is one of: PsO, AD, PsA and renal nephropathy.


SEQUENCE LISTING (313 sequences)

SEQ ID NO: 1 - protein, 177 aa, Homo sapiens
MKLQCVSLWLLGTILILCSVDNHGLRRCLISTDMHHIEESFQEIKRAIQAKDTFPNVTILSTLETLQIIKPLDVCCVTKNLLAFYVDRVFKDHQEPNPKILRKISSIANSFLYMQKTLRQCQEQRQCHCRQEATNATRVIHDNYDQLEVHAAAIKSLGELDVFLAWINKNHEVMFSA

SEQ ID NO: 2 - protein, 220 aa, synthetic construct
  REGION 1..220: note = synthetic construct
DIVMTQSPSSLAMSVGQKVTMSCKSSQSLLSSYNQKNYLAWYQQKPGQSPKLLVYFASTRPPGVPDRFIGSGSGSDFTLTISSVQAEDLADYFCQQHERLPITFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 17 aa, synthetic construct
  REGION 1..17: note = synthetic construct
KSSQSLLSSYNQKNYLA

SEQ ID NO: 4 - protein, 7 aa, synthetic construct
  REGION 1..7: note = synthetic construct
FASTRPP

SEQ ID NO: 5 - protein, 9 aa, synthetic construct
  REGION 1..9: note = synthetic construct
QQHERLPIT

SEQ ID NO: 6 - protein, 443 aa, synthetic construct
  REGION 1..443: note = synthetic construct
EVQLQQSGPVLVKPGASVKMSCKASGYKFTDYFVGWVKQSHGKSLEWIGYVDPENGYTRCNQKFKGKATLTVDKSSSTAYMELNSLTSEDSAVYYCARYDYDWAWFTNWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 7 - protein, 10 aa, synthetic construct
  REGION 1..10: note = synthetic construct
GYKFTDYFVG

SEQ ID NO: 8 - protein, 17 aa, synthetic construct
  REGION 1..17: note = synthetic construct
YVDPENGYTRCNQKFKG

SEQ ID NO: 9 - protein, 10 aa, synthetic construct
  REGION 1..10: note = synthetic construct
YDYDWAWFTN

SEQ ID NO: 10 - protein, 218 aa, synthetic construct
  REGION 1..218: note = synthetic construct
DIVLTQSPESLAVSLGQRATISCRASESVDNYGISFIHWYQQKPGQPPKLLIYRASNLKSGIPARFSGRGSRTDFTLTINPVETDDVATYYCQQINKDPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 11 - protein, 15 aa, synthetic construct
  REGION 1..15: note = synthetic construct
RASESVDNYGISFIH

SEQ ID NO: 12 - protein, 7 aa, synthetic construct
  REGION 1..7: note = synthetic construct
RASNLKS

SEQ ID NO: 13 - protein, 9 aa, synthetic construct
  REGION 1..9: note = synthetic construct
QQINKDPLT

SEQ ID NO: 14 - protein, 441 aa, synthetic construct
  REGION 1..441: note = synthetic construct
LVQLQQSDAELVKPGASLKISCKVSGYTFTDHALHWMKQRPEQGLEWIGYIYPRDGSTKYNEKFKGKATLTADRSSSTAYMELNSLTSEDSAVYFCARGWDFAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 15 - protein, 10 aa, synthetic construct
  REGION 1..10: note = synthetic construct
GYTFTDHALH

SEQ ID NO: 16 - protein, 17 aa, synthetic construct
  REGION 1..17: note = synthetic construct
YIYPRDGSTKYNEKFKG

SEQ ID NO: 17 - protein, 8 aa, synthetic construct
  REGION 1..8: note = synthetic construct
GWDFAMDY

SEQ ID NO: 18 - protein, 214 aa, synthetic construct
  REGION 1..214: note = synthetic construct
DIKMTQSPSSMYASLGERVTITCKASQDINSYLSWFQQKPGKSPKTLIYRANRLVDGVPSRFSGSGSGQDYSLTISSLEYEDMGIYFCLQYDDFPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - protein, 11 aa, synthetic construct
  REGION 1..11: note = synthetic construct
KASQDINSYLS

SEQ ID NO: 20 - protein, 7 aa, synthetic construct
  REGION 1..7: note = synthetic construct
RANRLVD

SEQ ID NO: 21 - protein, 9 aa, synthetic construct
  REGION 1..9: note = synthetic construct
LQYDDFPYT

SEQ ID NO: 22 - protein, 440 aa, synthetic construct
  REGION 1..440: note = synthetic construct
QIQLVQSGPELKKPGETVKISCKASGYTFTTYGMSWVKQAPGKGLKWMVWINTYSGVPTYVDDFKGRFAFSLETSASTAYLQINNLKNEDTATYFCARRDEGFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 23 - protein, 10 aa, synthetic construct
  REGION 1..10: note = synthetic construct
GYTFTTYGMS

SEQ ID NO: 24 - protein, 17 aa, synthetic construct
  REGION 1..17: note = synthetic construct
WINTYSGVPTYVDDFKG

SEQ ID NO: 25 - protein, 7 aa, synthetic construct
  REGION 1..7: note = synthetic construct
RDEGFAY

SEQ ID NO: 26 - protein, 220 aa, synthetic construct
  REGION 1..220: note = synthetic construct
DIVMTQSPSSLAMSVGQKVTMSCKSSQSLLSSYNQKNYLAWYQQKPGQSPKLLVYFASTRESGVPDRFIGSGSGSDFTLTISSVQAEDLADYFCQQHYSTPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 27 - protein, 17 aa, synthetic construct
  REGION 1..17: note = synthetic construct
KSSQSLLSSYNQKNYLA

SEQ ID NO: 28 - protein, 7 aa, synthetic construct
  REGION 1..7: note = synthetic construct
FASTRES

SEQ ID NO: 29 - protein, 9 aa, synthetic construct
  REGION 1..9: note = synthetic construct
QQHYSTPLT

SEQ ID NO: 30 - protein, 443 aa, synthetic construct
  REGION 1..443: note = synthetic construct
EVQLQQSGPVLVKPGASVKMSCKASGYTFTDYYMNWVKQSHGKSLEWIGFINPYNDDTRCNQKFKGKATLTVDKSSSTAYMELNSLTSEDSAVYYCARYDGYWAWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 31 - protein, 10 aa, synthetic construct
  REGION 1..10: note = synthetic construct
GYTFTDYYMN

SEQ ID NO: 32 - protein, 17 aa, synthetic construct
  REGION 1..17: note = synthetic construct
FINPYNDDTRCNQKFKG

SEQ ID NO: 33 - protein, 10 aa, synthetic construct
  REGION 1..10: note = synthetic construct
YDGYWAWFAY

SEQ ID NO: 34 - protein, 111 aa, synthetic construct
  REGION 1..111: note = synthetic construct
AIDMTQTPASVSEPVGGTVTIKCQASQSISSYLAWYQQKPGQPPKLLIYSASTLASGVPSRFRGSGSGREFTLTISDLECADAATYYCQCHYHGSSYWDNSFGGGTEVVVK

SEQ ID NO: 35 - protein, 11 aa, synthetic construct
  REGION 1..11: note = synthetic construct
QASQSISSYLA

SEQ ID NO: 36 - protein, 7 aa, synthetic construct
  REGION 1..7: note = synthetic construct
SASTLAS

SEQ ID NO: 37 - protein, 13 aa, synthetic construct
  REGION 1..13: note = synthetic construct
QCHYHGSSYWDNS

SEQ ID NO: 38 - protein, 120 aa, synthetic construct
  REGION 1..120: note = synthetic construct
QQLEESGGGLVQPEGSLTLTCTASGFSFSSSYWICWVRQAPGKGLEWLACIDTGVSGDTYYANWAEGRFTISKTSSTTVTLQMTSLTAADTATYFCARDIFGSAIDNSLWGPGTLVTVSS

SEQ ID NO: 39 - protein, 11 aa, synthetic construct
  REGION 1..11: note = synthetic construct
GFSFSSSYWIC

SEQ ID NO: 40 - protein, 18 aa, synthetic construct
  REGION 1..18: note = synthetic construct
CIDTGVSGDTYYANWAEG

SEQ ID NO: 41 - protein, 11 aa, synthetic construct
  REGION 1..11: note = synthetic construct
DIFGSAIDNSL

SEQ ID NO: 42 - protein, 112 aa, synthetic construct
  REGION 1..112: note = synthetic construct
ADVVMTQTPASVEAAVGGTVTIKCQASESIGNALAWYQQKPGQPPKLLIYDASKLASGVPSRFKGSGSGTQFTLTISDLECADAATYYCQCHYHGSSYWDNSFGGGTEVVVK

SEQ ID NO: 43 - protein, 11 aa, synthetic construct
  REGION 1..11: note = synthetic construct
QASESIGNALA

SEQ ID NO: 44 - protein, 7 aa, synthetic construct
  REGION 1..7: note = synthetic construct
DASKLAS

SEQ ID NO: 45 - protein, 13 aa, synthetic construct
  REGION 1..13: note = synthetic construct
QCHYHGSSYWDNS

SEQ ID NO: 46 - protein, 120 aa, synthetic construct
  REGION 1..120: note = synthetic construct
QQLEESGGGLVQPEGSLTLTCTASGFSFSSSYWICWVRQAPGKGLEWLACIDTGVSGDTYYANWAKGRFTISKTSSTTVTLQMTSLTAADTATYFCARDIFGSAIDNSLWGPGTLVTVSS

SEQ ID NO: 47 - protein, 11 aa, synthetic construct
  REGION 1..11: note = synthetic construct
GFSFSSSYWIC

SEQ ID NO: 48 - protein, 18 aa, synthetic construct
  REGION 1..18: note = synthetic construct
CIDTGVSGDTYYANWAKG

SEQ ID NO: 49 - protein, 11 aa, synthetic construct
  REGION 1..11: note = synthetic construct
DIFGSAIDNSL

SEQ ID NO: 50 - protein, 112 aa, synthetic construct
  REGION 1..112: note = synthetic construct
ADVVMTQTPSSVEAAVGGTVTIKCQASQSIGSSLAWYQQKPGQRPKLLIYGASTLASGVPSRFKGSGSGTEFTLTISDLECADAATYYCQCHYHGSSYWDNSFGGGTEVVVK

SEQ ID NO: 51 - protein, 11 aa, synthetic construct
  REGION 1..11: note = synthetic construct
QASQSIGSSLA

SEQ ID NO: 52 - protein, 7 aa, synthetic construct
  REGION 1..7: note = synthetic construct
GASTLAS

SEQ ID NO: 53 - protein, 13 aa, synthetic construct
  REGION 1..13: note = synthetic construct
QCHYHGSSYWDNS

SEQ ID NO: 54 - protein, 121 aa, synthetic construct
  REGION 1..121: note = synthetic construct
QCQSLEESGGGLVEPEGSLTLTCKASGFSFSSSYWICWVRQAPGKGLEWIACTDTFSGDTYYASWAKGRFTISKTSSTTVTLRMTSLTDADTATYFCARDIFGTAVHISLWGPGTLVTVSS

SEQ ID NO: 55 - protein, 11 aa, synthetic construct
  REGION 1..11: note = synthetic construct
GFSFSSSYWIC

SEQ ID NO: 56 - protein, 17 aa, synthetic construct
  REGION 1..17: note = synthetic construct
CTDTFSGDTYYASWAKG

SEQ ID NO: 57 - protein, 11 aa, synthetic construct
  REGION 1..11: note = synthetic construct
DIFGTAVHISL

SEQ ID NO: 58 - protein, 111 aa, synthetic construct
  REGION 1..111: note = synthetic construct
ADVVMTQTPSSVSEPVGGTVTIKCQASQSIYSYLSWYQQKPGQPPKLLIYEASKLASGVPSRFKGSGSGTQFTLTISGVECADAATYYCQQGYSSSNVDNTFGGGTEVVVT

SEQ ID NO: 59 - protein, 11 aa, synthetic construct
  REGION 1..11: note = synthetic construct
QASQSIYSYLS

SEQ ID NO: 60 - protein, 7 aa, synthetic construct
  REGION 1..7: note = synthetic construct
EASKLAS

SEQ ID NO: 61 - protein, 12 aa, synthetic construct
  REGION 1..12: note = synthetic construct
QQGYSSSNVDNT

SEQ ID NO: 62 - protein, 119 aa, synthetic construct
  REGION 1..119: note = synthetic construct
QCQSVEESGGRLVTPGTPLTLTCTVSGIDLSIYAMGWVRQAPGKGLEYIGIIDTTGTAYYARWAKGRFTISKTSATVALKITSPTTEDTATYFCARDSGSLYYGSYALWGPGTLVTVSS

SEQ ID NO: 63 - protein, 10 aa, synthetic construct
  REGION 1..10: note = synthetic construct
GIDLSIYAMG

SEQ ID NO: 64 - protein, 16 aa, synthetic construct
  REGION 1..16: note = synthetic construct
IIDTTGTAYYARWAKG

SEQ ID NO: 65 - protein, 12 aa, synthetic construct
  REGION 1..12: note = synthetic construct
DSGSLYYGSYAL

SEQ ID NO: 66 - protein, 109 aa, synthetic construct
  REGION 1..109: note = synthetic construct
EIVMTQSPGTLSLSPGERATLSCRASQSISSGYLAWYQQKPGQAPRLLIYGASSRATGIPDRFSGSGSGTDFTLTISRLEPEDFATYYCLQFKSFPLTFGQGTKLEIKR

SEQ ID NO: 67 - protein, 12 aa, synthetic construct
  REGION 1..12: note = synthetic construct
RASQSISSGYLA

SEQ ID NO: 68 - protein, 7 aa, synthetic construct
  REGION 1..7: note = synthetic construct
GASSRAT

SEQ ID NO: 69 - protein, 9 aa, synthetic construct
  REGION 1..9: note = synthetic construct
LQFKSFPLT

SEQ ID NO: 70 - protein, 119 aa, synthetic construct
  REGION 1..119: note = synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGGDYGDMGYWGQGTLVTVSS

SEQ ID NO: 71 - protein, 10 aa, synthetic construct
  REGION 1..10: note = synthetic construct
GGTFSSYAIS

SEQ ID NO: 72 - protein, 17 aa, synthetic construct
  REGION 1..17: note = synthetic construct
GIIPIFGTANYAQKFQG

SEQ ID NO: 73 - protein, 10 aa, synthetic construct
  REGION 1..10: note = synthetic construct
DGGDYGDMGY

SEQ ID NO: 74 - protein, 114 aa, synthetic construct
  REGION 1..114: note = synthetic construct
EIVMTQSPDSLAVSLGERATINCKSSQSVLYRSNSKSYLAWYQQKPGQPPKLLIYWASTRESGVPDRFSGSGSGTDFTLTISSLQAEDVAVYYCQQYYSTPGSFGPGTKVEIKR

SEQ ID NO: 75 - protein, 17 aa, synthetic construct
  REGION 1..17: note = synthetic construct
KSSQSVLYRSNSKSYLA

SEQ ID NO: 76 - protein, 7 aa, synthetic construct
  REGION 1..7: note = synthetic construct
WASTRES

SEQ ID NO: 77 - protein, 9 aa, synthetic construct
  REGION 1..9: note = synthetic construct
QQYYSTPGS

SEQ ID NO: 78 - protein, 126 aa, synthetic construct
  REGION 1..126: note = synthetic construct
EVQLLESGGGLVQPGGSLRLSCAASGFTFSSYAMSWVRQAPGKGLEWVSAISQSGGSTYYADSVKGRFTISRDNSKNTLYLQMNSLRAEDTAVYYCARHPERGSRRGPYYYYMDVWGKGTTVTVSS

SEQ ID NO: 79 - protein, 10 aa, synthetic construct
  REGION 1..10: note = synthetic construct
GFTFSSYAMS

SEQ ID NO: 80 - protein, 17 aa, synthetic construct
  REGION 1..17: note = synthetic construct
AISQSGGSTYYADSVKG

SEQ ID NO: 81 - protein, 17 aa, synthetic construct
  REGION 1..17: note = synthetic construct
HPERGSRRGPYYYYMDV

SEQ ID NO: 82 - protein, 109 aa, synthetic construct
  REGION 1..109: note = synthetic construct
DIQLTQSPSSLSASLGDRVTITCRASQGISSWLAWYQQKPGKAPKLLIYAASTLQSGVPSRFSGSGSGTDFTLTISSLQPEDVATYYCQQYYDYSPWAFGQGTKLEIKR

SEQ ID NO: 83 - protein, 11 aa, synthetic construct
  REGION 1..11: note = synthetic construct
RASQGISSWLA

SEQ ID NO: 84 - protein, 7 aa, synthetic construct
  REGION 1..7: note = synthetic construct
AASTLQS

SEQ ID NO: 85 - protein, 10 aa, synthetic construct
  REGION 1..10: note = synthetic construct
QQYYDYSPWA

SEQ ID NO: 86 - protein, 125 aa, synthetic construct
  REGION 1..125: note = synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGLSLLYYDLSENYFDYWGQGTTVTVSS

SEQ ID NO: 87 - protein, 10 aa, synthetic construct
  REGION 1..10: note = synthetic construct
GFTFSSYAMS

SEQ ID NO: 88 - protein, 17 aa, synthetic construct
  REGION 1..17: note = synthetic construct
AISGSGGSTYYADSVKG

SEQ ID NO: 89 - protein, 16 aa, synthetic construct
  REGION 1..16: note = synthetic construct
GLSLLYYDLSENYFDY

SEQ ID NO: 90 - protein, 11 aa, synthetic construct
  REGION 1..11: note = synthetic construct
QASQSIYSYLS

SEQ ID NO: 91 - protein, 7 aa, synthetic construct
  REGION 1..7: note = synthetic construct
EASKLAS

SEQ ID NO: 92 - protein, 12 aa, synthetic construct
  REGION 1..12: note = synthetic construct
QQGYSSSNVDNT

SEQ ID NO: 93 - protein, 10 aa, synthetic construct
  REGION 1..10: note = synthetic construct
GIDLSIYAMG

SEQ ID NO: 94 - protein, 16 aa, synthetic construct
  REGION 1..16: note = synthetic construct
IIDTTGTAYYARWAKG

SEQ ID NO: 95 - protein, 12 aa, synthetic construct
  REGION 1..12: note = synthetic construct
DSGSLYYGSYAL

SEQ ID NO: 96 - protein, 11 aa, synthetic construct
  REGION 1..11: note = synthetic construct
QASESIYSSLA

SEQ ID NO: 97 - protein, 7 aa, synthetic construct
  REGION 1..7: note = synthetic construct
GASTLAS

SEQ ID NO: 98 - protein, 13 aa, synthetic construct
  REGION 1..13: note = synthetic construct
QCHYHGSSYWDNS

SEQ ID NO: 99 - protein, 11 aa, synthetic construct
  REGION 1..11: note = synthetic construct
GFSFSSSYWIC

SEQ ID NO: 100 - protein, 17 aa, synthetic construct
  REGION 1..17: note = synthetic construct
CTDTFSGDTYYASWAKG

SEQ ID NO: 101 - protein, 11 aa, synthetic construct
  REGION 1..11: note = synthetic construct
DIFGTAVHISL

SEQ ID NO: 102 - protein, 11 aa, synthetic construct
  REGION 1..11: note = synthetic construct
QASESIYSSLA

SEQ ID NO: 103 - protein, 7 aa, synthetic construct
  REGION 1..7: note = synthetic construct
GASTLAS

SEQ ID NO: 104 - protein, 13 aa, synthetic construct
  REGION 1..13: note = synthetic construct
QCHYHGSSYWDNS

SEQ ID NO: 105 - protein, 11 aa, synthetic construct
  REGION 1..11: note = synthetic construct
GFSFSSSYWIC

SEQ ID NO: 106 - protein, 17 aa, synthetic construct
  REGION 1..17: note = synthetic construct
CTDTFSGDTYYASWAKG

SEQ ID NO: 107 - protein, 11 aa, synthetic construct
  REGION 1..11: note = synthetic construct
DIFGTAVHISL

SEQ ID NO: 108 - protein, 11 aa, synthetic construct
  REGION 1..11: note = synthetic construct
QASQSIGSSLA

SEQ ID NO: 109 - protein, 7 aa, synthetic construct
  REGION 1..7: note = synthetic construct
GASTLAS

SEQ ID NO: 110 - protein, 13 aa, synthetic construct
  REGION 1..13: note = synthetic construct
QCHYHGSSYWDNS

SEQ ID NO: 111 - protein, 11 aa, synthetic construct
  REGION 1..11: note = synthetic construct
GFSFSSSYWIC

SEQ ID NO: 112 - protein, 17 aa, synthetic construct
  REGION 1..17: note = synthetic construct
CTDTFSGDTYYASWAKG

SEQ ID NO: 113 - protein, 11 aa, synthetic construct
  REGION 1..11: note = synthetic construct
DIFGTAVHISL

SEQ ID NO: 114 - protein, 11 aa, synthetic construct
  REGION 1..11: note = synthetic construct
QASESIYSSLA

SEQ ID NO: 115 - protein, 7 aa, synthetic construct
  REGION 1..7: note = synthetic construct
GASTLAS

SEQ ID NO: 116 - protein, 13 aa, synthetic construct
  REGION 1..13: note = synthetic construct
QCHYHGSSYWDNS

SEQ ID NO: 117 - protein, 11 aa, synthetic construct
  REGION 1..11: note = synthetic construct
GFSFSSSYWIC

SEQ ID NO: 118 - protein, 17 aa, synthetic construct
  REGION 1..17: note = synthetic construct
CTDTFSGDTYYASWAKG

SEQ ID NO: 119 - protein, 11 aa, synthetic construct
  REGION 1..11: note = synthetic construct
DIFGTAVHISL

SEQ ID NO: 120 - protein, 11 aa, synthetic construct
  REGION 1..11: note = synthetic construct
QASQSISSYLA

SEQ ID NO: 121 - protein, 7 aa, synthetic construct
  REGION 1..7: note = synthetic construct
GASTLAS

SEQ ID NO: 122 - protein, 13 aa, synthetic construct
  REGION 1..13: note = synthetic construct
QCHYHGSSYWDNS

SEQ ID NO: 123 - protein, 11 aa, synthetic construct
  REGION 1..11: note = synthetic construct
GFSFSSSYWIC

SEQ ID NO: 124 - protein, 17 aa, synthetic construct
  REGION 1..17: note = synthetic construct
CTDTFSGDTYYASWAKG

SEQ ID NO: 125 - protein, 11 aa, synthetic construct
  REGION 1..11: note = synthetic construct
DIFGTAVHISL

SEQ ID NO: 126 - protein, 11 aa, synthetic construct
  REGION 1..11: note = synthetic construct
QASQSISSYLA

SEQ ID NO: 127 - protein, 7 aa, synthetic construct
  REGION 1..7: note = synthetic construct
GASTLAS

SEQ ID NO: 128 - protein, 14 aa, synthetic construct
  REGION 1..14: note = synthetic construct
QSAVYSSSSGYGVP

SEQ ID NO: 129 - protein, 10 aa, synthetic construct
  REGION 1..10: note = synthetic construct
GFSLSSYDMS

SEQ ID NO: 130 - protein, 16 aa, synthetic construct
  REGION 1..16: note = synthetic construct
IIDSIGSIWYANWAKG

SEQ ID NO: 131 - protein, 11 aa, synthetic construct
  REGION 1..11: note = synthetic construct
ESGPINTDYDL

SEQ ID NO: 132 - protein, 13 aa, synthetic construct
  REGION 1..13: note = synthetic construct
QTSESFYSNNILS

SEQ ID NO: 133 - protein, 7 aa, synthetic construct
  REGION 1..7: note = synthetic construct
EASKLAS

SEQ ID NO: 134 - protein, 12 aa, synthetic construct
  REGION 1..12: note = synthetic construct
QSAIYDGSYIVT

SEQ ID NO: 135 - protein, 10 aa, synthetic construct
  REGION 1..10: note = synthetic construct
GFSLSSYDMS

SEQ ID NO: 136 - protein, 16 aa, synthetic construct
  REGION 1..16: note = synthetic construct
IIDSIGSIWYANWAKG

SEQ ID NO: 137 - protein, 11 aa, synthetic construct
  REGION 1..11: note = synthetic construct
ESGPINTDYDL

SEQ ID NO: 138 - protein, 11 aa, synthetic construct
  REGION 1..11: note = synthetic construct
QASQSISSYLA

SEQ ID NO: 139 - protein, 7 aa, synthetic construct
  REGION 1..7: note = synthetic construct
GASTLAS

SEQ ID NO: 140 - protein, 14 aa, synthetic construct
  REGION 1..14: note = synthetic construct
QSAVYSSSSGYGVP

SEQ ID NO: 141 - protein, 10 aa, synthetic construct
  REGION 1..10: note = synthetic construct
GFSLSSYDMS

SEQ ID NO: 142 - protein, 16 aa, synthetic construct
  REGION 1..16: note = synthetic construct
IIDSIGSIWYANWAKG

SEQ ID NO: 143 - protein, 11 aa, synthetic construct
  REGION 1..11: note = synthetic construct
ESGPINTDYDL

SEQ ID NO: 144 - protein, 11 aa, synthetic construct
  REGION 1..11: note = synthetic construct
QASQSISSYLA

SEQ ID NO: 145 - protein, 7 aa, synthetic construct
  REGION 1..7: note = synthetic construct
GASTLAS

SEQ ID NO: 146 - protein, 14 aa, synthetic construct
  REGION 1..14: note = synthetic construct
QSAVYSSSSGYGVP

SEQ ID NO: 147 - protein, 10 aa, synthetic construct
  REGION 1..10: note = synthetic construct
GFSLSSYDMS

SEQ ID NO: 148 - protein, 16 aa, synthetic construct
  REGION 1..16: note = synthetic construct
IIDSIGSIWYANWAKG

SEQ ID NO: 149 - protein, 11 aa, synthetic construct
  REGION 1..11: note = synthetic construct
ESGPINTDYDL

SEQ ID NO: 150 - protein, 11 aa, synthetic construct
  REGION 1..11: note = synthetic construct
QASESISSYLA

SEQ ID NO: 151 - protein, 7 aa, synthetic construct
  REGION 1..7: note = synthetic construct
GASTLAS

SEQ ID NO: 152 - protein, 10 aa, synthetic construct
  REGION 1..10: note = synthetic construct
QGYFGDYIYG

SEQ ID NO: 153 - protein, 10 aa, synthetic construct
  REGION 1..10: note = synthetic construct
GFSLTSYAMS

SEQ ID NO: 154 - protein, 16 aa, synthetic construct
  REGION 1..16: note = synthetic construct
IIGSPGTTGYATWAKG

SEQ ID NO: 155 - protein, 9 aa, synthetic construct
  REGION 1..9: note = synthetic construct
GWFYYGMAL

SEQ ID NO: 156 - protein, 8 aa, synthetic construct
  REGION 1..8: note = synthetic construct
  SITE 2: note = misc_feature - Xaa can be any naturally occurring amino acid
PXQEHLTP

SEQ ID NO: 157 - protein, 7 aa, synthetic construct
  REGION 1..7: note = synthetic construct
  SITE 7: note = misc_feature - Xaa can be any naturally occurring amino acid
GASTLAX

SEQ ID NO: 158 - protein, 10 aa, synthetic construct
  REGION 1..10: note = synthetic construct
QGYFGDYIYG

SEQ ID NO: 159 - protein, 10 aa, synthetic construct
  REGION 1..10: note = synthetic construct
GFSLTSYAMS

SEQ ID NO: 160 - protein, 16 aa, synthetic construct
  REGION 1..16: note = synthetic construct
IIGSPGTTGYATWAKG

SEQ ID NO: 161 - protein, 9 aa, synthetic construct
  REGION 1..9: note = synthetic construct
GWFYYGMAL

SEQ ID NO: 162 - protein, 11 aa, synthetic construct
  REGION 1..11: note = synthetic construct
QASESISSYLA

SEQ ID NO: 163 - protein, 7 aa, synthetic construct
  REGION 1..7: note = synthetic construct
GASTLAS

SEQ ID NO: 164 - protein, 10 aa, synthetic construct
  REGION 1..10: note = synthetic construct
QGYFGDYIYG

SEQ ID NO: 165 - protein, 10 aa, synthetic construct
  REGION 1..10: note = synthetic construct
GFSLTSYAMS

SEQ ID NO: 166 - protein, 16 aa, synthetic construct
  REGION 1..16: note = synthetic construct
IIGSPGTTGYATWAKG

SEQ ID NO: 167 - protein, 9 aa, synthetic construct
  REGION 1..9: note = synthetic construct
GWFYYGMAL

SEQ ID NO: 168 - protein, 11 aa, synthetic construct
  REGION 1..11: note = synthetic construct
QASESISSYLA

SEQ ID NO: 169 - protein, 7 aa, synthetic construct
  REGION 1..7: note = synthetic construct
GASTLAS

SEQ ID NO: 170 - protein, 10 aa, synthetic construct
  REGION 1..10: note = synthetic construct
QGYFGDYIYG

SEQ ID NO: 171 - protein, 10 aa, synthetic construct
  REGION 1..10: note = synthetic construct
GFSLTSYAMS

SEQ ID NO: 172 - protein, 16 aa, synthetic construct
  REGION 1..16: note = synthetic construct
IIGSPGTTGYATWAKG

SEQ ID NO: 173 - protein, 9 aa, synthetic construct
  REGION 1..9: note = synthetic construct
GWFYYGMAL

SEQ ID NO: 174 - protein, 11 aa, synthetic construct
  REGION 1..11: note = synthetic construct
QASESISSYLA

SEQ ID NO: 175 - protein, 7 aa, synthetic construct
  REGION 1..7: note = synthetic construct
GASTLAS

SEQ ID NO: 176 - protein, 10 aa, synthetic construct
  REGION 1..10: note = synthetic construct
QGYFGDYIYG

SEQ ID NO: 177 - protein, 10 aa, synthetic construct
  REGION 1..10: note = synthetic construct
GFSLTSYAMS

SEQ ID NO: 178 - protein, 16 aa, synthetic construct
  REGION 1..16: note = synthetic construct
IIGSPGTTGYATWAKG

SEQ ID NO: 179 - protein, 9 aa, synthetic construct
  REGION 1..9: note = synthetic construct
GWFYYGMAL

SEQ ID NO: 180 - protein, 11 aa, synthetic construct
  REGION 1..11: note = synthetic construct
QASESISSYLA

SEQ ID NO: 181 - protein, 7 aa, synthetic construct
  REGION 1..7: note = synthetic construct
GASTLAS

SEQ ID NO: 182 - protein, 10 aa, synthetic construct
  REGION 1..10: note = synthetic construct
QGYFGDYIYG

SEQ ID NO: 183 - protein, 10 aa, synthetic construct
  REGION 1..10: note = synthetic construct
GFSLTSYAMS

SEQ ID NO: 184 - protein, 16 aa, synthetic construct
  REGION 1..16: note = synthetic construct
IIGSPGTTGYATWAKG

SEQ ID NO: 185 - protein, 9 aa, synthetic construct
  REGION 1..9: note = synthetic construct
GWFYYGMAL

SEQ ID NO: 186 - protein, 11 aa, synthetic construct
  REGION 1..11: note = synthetic construct
QASQSISNYLS

SEQ ID NO: 187 - protein, 7 aa, synthetic construct
  REGION 1..7: note = synthetic construct
WASNLAS

SEQ ID NO: 188 - protein, 13 aa, synthetic construct
  REGION 1..13: note = synthetic construct
LGEFSCSSADCFA

SEQ ID NO: 189 - protein, 10 aa, synthetic construct
  REGION 1..10: note = synthetic construct
GFDLSNYAMT

SEQ ID NO: 190 - protein, 16 aa, synthetic construct
  REGION 1..16: note = synthetic construct
AIHGSGVTDCASWTRG

SEQ ID NO: 191 - protein, 11 aa, synthetic construct
  REGION 1..11: note = synthetic construct
ESAGINTDYDL

SEQ ID NO: 192 - protein, 11 aa, synthetic construct
  REGION 1..11: note = synthetic construct
QASQSISNYLS

SEQ ID NO: 193 - protein, 7 aa, synthetic construct
  REGION 1..7: note = synthetic construct
WASNLAS

SEQ ID NO: 194 - protein, 13 aa, synthetic construct
  REGION 1..13: note = synthetic construct
LGEFSCSSADCFA

SEQ ID NO: 195 - protein, 10 aa, synthetic construct
  REGION 1..10: note = synthetic construct
GFDLSNYAMT

SEQ ID NO: 196 - protein, 16 aa, synthetic construct
  REGION 1..16: note = synthetic construct
AIHGSGVTDCASWTRG

SEQ ID NO: 197 - protein, 11 aa, synthetic construct
  REGION 1..11: note = synthetic construct
ESAGINTDYDL

SEQ ID NO: 198 - protein, 11 aa, synthetic construct
  REGION 1..11: note = synthetic construct
QASKSIGSYLS

SEQ ID NO: 199 - protein, 7 aa, synthetic construct
  REGION 1..7: note = synthetic construct
RASTLAS

SEQ ID NO: 200 - protein, 10 aa, synthetic construct
  REGION 1..10: note = synthetic construct
QAYYGDYIYD

SEQ ID NO: 201 - protein, 10 aa, synthetic construct
  REGION 1..10: note = synthetic construct
GFSLSSYPMS

SEQ ID NO: 202 - protein, 16 aa, synthetic construct
  REGION 1..16: note = synthetic construct
IIGSTGSTGYANWAKG

SEQ ID NO: 203 - protein, 9 aa, synthetic construct
  REGION 1..9: note = synthetic construct
GWFYYGLDL

SEQ ID NO: 204 - protein, 11 aa, synthetic construct
  REGION 1..11: note = synthetic construct
QASQSIGSYLS

SEQ ID NO: 205 - protein, 7 aa, synthetic construct
  REGION 1..7: note = synthetic construct
RASTLAS

SEQ ID NO: 206 - protein, 10 aa, synthetic construct
  REGION 1..10: note = synthetic construct
QAYYGDYIYD

SEQ ID NO: 207 - protein, 10 aa, synthetic construct
  REGION 1..10: note = synthetic construct
GFSLSSYPMS

SEQ ID NO: 208 - protein, 16 aa, synthetic construct
  REGION 1..16: note = synthetic construct
IIGSTGSTGYANWAKG

SEQ ID NO: 209 - protein, 9 aa, synthetic construct
  REGION 1..9: note = synthetic construct
GWFYYGLDL

SEQ ID NO: 210 - protein, 11 aa, synthetic construct
  REGION 1..11: note = synthetic construct
QASQSIGSYLA

SEQ ID NO: 211 - protein, 7 aa, synthetic construct
  REGION 1..7: note = synthetic construct
DASDLAS

SEQ ID NO: 212 - protein, 12 aa, synthetic construct
  REGION 1..12: note = synthetic construct
QSAVYDSSYVVT

SEQ ID NO: 213 - protein, 10 aa, synthetic construct
  REGION 1..10: note = synthetic construct
GFSLSSYDMS

SEQ ID NO: 214 - protein, 16 aa, synthetic construct
  REGION 1..16: note = synthetic construct
ISDSLGTTWYANWAKG

SEQ ID NO: 215 - protein, 11 aa, synthetic construct
  REGION 1..11: note = synthetic construct
ESGDIRIDYDL

SEQ ID NO: 216 - protein, 11 aa, synthetic construct
  REGION 1..11: note = synthetic construct
QASQSIGSYLA

SEQ ID NO: 217 - protein, 7 aa, synthetic construct
  REGION 1..7: note = synthetic construct
DASDLAS

SEQ ID NO: 218 - protein, 12 aa, synthetic construct
  REGION 1..12: note = synthetic construct
QSAVYDSSYVVT

SEQ ID NO: 219 - protein, 10 aa, synthetic construct
  REGION 1..10: note = synthetic construct
GFSLSSYDMS

SEQ ID NO: 220 - protein, 16 aa, synthetic construct
  REGION 1..16: note = synthetic construct
ISDSLGTTWYANWAKG

SEQ ID NO: 221 - protein, 11 aa, synthetic construct
  REGION 1..11: note = synthetic construct
ESGDIRIDYDL

SEQ ID NO: 222 - protein, 11 aa, synthetic construct
  REGION 1..11: note = synthetic construct
QASQSIGSYLA

SEQ ID NO: 223 - protein, 7 aa, synthetic construct
  REGION 1..7: note = synthetic construct
DASDLAS

SEQ ID NO: 224 - protein, 12 aa, synthetic construct
  REGION 1..12: note = synthetic construct
QSAVYDSSYVVT

SEQ ID NO: 225 - protein, 10 aa, synthetic construct
  REGION 1..10: note = synthetic construct
GFSLSSYDMS

SEQ ID NO: 226 - protein, 16 aa, synthetic construct
  REGION 1..16: note = synthetic construct
ISDSLGTTWYANWAKG

SEQ ID NO: 227 - protein, 11 aa, synthetic construct
  REGION 1..11: note = synthetic construct
ESGDIRIDYDL

SEQ ID NO: 228 - protein, 11 aa, synthetic construct
  REGION 1..11: note = synthetic construct
QASQSIGSYLA

SEQ ID NO: 229 - protein, 7 aa, synthetic construct
  REGION 1..7: note = synthetic construct
DASDLAS

SEQ ID NO: 230 - protein, 12 aa, synthetic construct
  REGION 1..12: note = synthetic construct
QSAVYDSSYVVT

SEQ ID NO: 231 - protein, 10 aa, synthetic construct
  REGION 1..10: note = synthetic construct
GFSLSSYDMS

SEQ ID NO: 232 - protein, 16 aa, synthetic construct
  REGION 1..16: note = synthetic construct
ISDSLGTTWYANWAKG

SEQ ID NO: 233 - protein, 11 aa, synthetic construct
  REGION 1..11: note = synthetic construct
ESGDIRIDYDL

SEQ ID NO: 234 - protein, 12 aa, synthetic construct
  REGION 1..12: note = synthetic construct
QASQSLNINYLS

SEQ ID NO: 235 - protein, 7 aa, synthetic construct
  REGION 1..7: note = synthetic construct
SASTLAS

SEQ ID NO: 236 - protein, 12 aa, synthetic construct
  REGION 1..12: note = synthetic construct
QQSVNYGRVDNI

SEQ ID NO: 237 - protein, 10 aa, synthetic construct
  REGION 1..10: note = synthetic construct
GFTISNYHMS

SEQ ID NO: 238 - protein, 16 aa, synthetic construct
  REGION 1..16: note = synthetic construct
RIVSYGGTYYANWAKG

SEQ ID NO: 239 - protein, 11 aa, synthetic construct
  REGION 1..11: note = synthetic construct
DSSGSGFSFAL

SEQ ID NO: 240 - protein, 12 aa, synthetic construct
  REGION 1..12: note = synthetic construct
QASQSITTSYLS

SEQ ID NO: 241 - protein, 7 aa, synthetic construct
  REGION 1..7: note = synthetic construct
KASTLAS

SEQ ID NO: 242 - protein, 12 aa, synthetic construct
  REGION 1..12: note = synthetic construct
QQSHNYGRVDNI

SEQ ID NO: 243 - protein, 10 aa, synthetic construct
  REGION 1..10: note = synthetic construct
GFTISNYHMS

SEQ ID NO: 244 - protein, 16 aa, synthetic construct
  REGION 1..16: note = synthetic construct
RIVSYGGTYYANWAKG

SEQ ID NO: 245 - protein, 11 aa, synthetic construct
  REGION 1..11: note = synthetic construct
DSSGSGFSFAL

SEQ ID NO: 246 - protein, 12 aa, synthetic construct
  REGION 1..12: note = synthetic construct
QASQSITTSYLS

SEQ ID NO: 247 - protein, 7 aa, synthetic construct
  REGION 1..7: note = synthetic construct
KASTLAS

SEQ ID NO: 248 - protein, 12 aa, synthetic construct
  REGION 1..12: note = synthetic construct
QQSHNYGRVDNI

SEQ ID NO: 249 - protein, 10 aa, synthetic construct
  REGION 1..10: note = synthetic construct
GFTISNYHMS

SEQ ID NO: 250 - protein, 16 aa, synthetic construct
  REGION 1..16: note = synthetic construct
RIVSYGGTYYANWAKG

SEQ ID NO: 251 - protein, 11 aa, synthetic construct
  REGION 1..11: note = synthetic construct
DSSGSGFSFAL

SEQ ID NO: 252 - protein, 12 aa, synthetic construct
  REGION 1..12: note = synthetic construct
QASQSITTSYLS

SEQ ID NO: 253 - protein, 7 aa, synthetic construct
  REGION 1..7: note = synthetic construct
KASTLAS

SEQ ID NO: 254 - protein, 12 aa, synthetic construct
  REGION 1..12: note = synthetic construct
QQSHNYGRVDNI

SEQ ID NO: 255 - protein, 10 aa, synthetic construct
  REGION 1..10: note = synthetic construct
GFTISNYHMS

SEQ ID NO: 256 - protein, 16 aa, synthetic construct
  REGION 1..16: note = synthetic construct
RIVSYGGTYYANWAKG

SEQ ID NO: 257 - protein, 11 aa, synthetic construct
  REGION 1..11: note = synthetic construct
DSSGSGFSFAL

SEQ ID NO: 258 - protein, 12 aa, synthetic construct
  REGION 1..12: note = synthetic construct
QASQSITTSYLS

SEQ ID NO: 259 - protein, 7 aa, synthetic construct
  REGION 1..7: note = synthetic construct
KASTLAS

SEQ ID NO: 260 - protein, 12 aa, synthetic construct
  REGION 1..12: note = synthetic construct
QQSHNYGRVDNI

SEQ ID NO: 261 - protein, 10 aa, synthetic construct
  REGION 1..10: note = synthetic construct
GFTISNYHMS

SEQ ID NO: 262 - protein, 16 aa, synthetic construct
  REGION 1..16: note = synthetic construct
RIVSYGGTYYANWAKG

SEQ ID NO: 263 - protein, 11 aa, synthetic construct
  REGION 1..11: note = synthetic construct
DSSGSGFSFAL

SEQ ID NO: 264 - protein, 11 aa, synthetic construct
  REGION 1..11: note = synthetic construct
QASQNIYSNLA

SEQ ID NO: 265 - protein, 7 aa, synthetic construct
  REGION 1..7: note = synthetic construct
KASTLAS

SEQ ID NO: 266 - protein, 10 aa, synthetic construct
  REGION 1..10: note = synthetic construct
QAYVGNYIYT

SEQ ID NO: 267 - protein, 10 aa, synthetic construct
  REGION 1..10: note = synthetic construct
GFSLSSYPMS

SEQ ID NO: 268 - protein, 16 aa, synthetic construct
  REGION 1..16: note = synthetic construct
IIGSSGSIGYASWAKG

SEQ ID NO: 269 - protein, 9 aa, synthetic construct
  REGION 1..9: note = synthetic construct
GWFYYGMDL

SEQ ID NO: 270 - protein, 11 aa, synthetic construct
  REGION 1..11: note = synthetic construct
QASQSIGSNLA

SEQ ID NO: 271 - protein, 7 aa, synthetic construct
  REGION 1..7: note = synthetic construct
RASTLAS

SEQ ID NO: 272 - protein, 10 aa, synthetic construct
  REGION 1..10: note = synthetic construct
QGYYGDGIYG

SEQ ID NO: 273 - protein, 10 aa, synthetic construct
  REGION 1..10: note = synthetic construct
GFSLSSYPMS

SEQ ID NO: 274 - protein, 16 aa, synthetic construct
  REGION 1..16: note = synthetic construct
LIGSSGTIYFATWAKG

SEQ ID NO: 275 - protein, 9 aa, synthetic construct
  REGION 1..9: note = synthetic construct
GWFYYGMDL

SEQ ID NO: 276 - protein, 11 aa, synthetic construct
  REGION 1..11: note = synthetic construct
QASQSISSWLS

SEQ ID NO: 277 - protein, 7 aa, synthetic construct
  REGION 1..7: note = synthetic construct
GASTLAS

SEQ ID NO: 278 - protein, 12 aa, synthetic construct
  REGION 1..12: note = synthetic construct
QSYYYISSSSYT

SEQ ID NO: 279 - protein, 10 aa, synthetic construct
  REGION 1..10: note = synthetic construct
GFSLSSYPMS

SEQ ID NO: 280 - protein, 16 aa, synthetic construct
  REGION 1..16: note = synthetic construct
LIGSSGTIYFATWAKG

SEQ ID NO: 281 - protein, 9 aa, synthetic construct
  REGION 1..9: note = synthetic construct
GWFYYGMDL

SEQ ID NO: 282 - protein, 11 aa, synthetic construct
  REGION 1..11: note = synthetic construct
QASQSISSYLA

SEQ ID NO: 283 - protein, 7 aa, synthetic construct
  REGION 1..7: note = synthetic construct
SASTLAS

SEQ ID NO: 284 - protein, 13 aa, synthetic construct
  REGION 1..13: note = synthetic construct
QCHYHGSSYWDNS

SEQ ID NO: 285 - protein, 11 aa, synthetic construct
  REGION 1..11: note = synthetic construct
GFSFSSSYWIC

SEQ ID NO: 286 - protein, 18 aa, synthetic construct
  REGION 1..18: note = synthetic construct
CIDTGVSGDTYYANWAEG

SEQ ID NO: 287 - protein, 11 aa, synthetic construct
  REGION 1..11: note = synthetic construct
DIFGSAIDNSL

SEQ ID NO: 288 - protein, 11 aa, synthetic construct
  REGION 1..11: note = synthetic construct
QASQNIYSNLA

SEQ ID NO: 289 - protein, 7 aa, synthetic construct
  REGION 1..7: note = synthetic construct
GASNLES

SEQ ID NO: 290 - protein, 10 aa, synthetic construct
  REGION 1..10: note = synthetic construct
QGGYSGGIYA

SEQ ID NO: 291 - protein, 10 aa, synthetic construct
  REGION 1..10: note = synthetic construct
GFSLTRHAMS

SEQ ID NO: 292 - protein, 16 aa, synthetic construct
  REGION 1..16: note = synthetic construct
AIHGSGVTDYASWAKG

SEQ ID NO: 293 - protein, 4 aa, synthetic construct
  REGION 1..4: note = synthetic construct
GSGL

SEQ ID NO: 294 - protein, 112 aa, synthetic construct
  REGION 1..112: note = synthetic construct
DIVMTQTPASVEAAVGGTVTIKCQASQSIYRYLSWYQQKPGQRPKLLIYGASNLASGVSSRFKGSGSGTEFTLTISDLECADAATYYCQSYYYSSSSGYGNVFGGGTEVVVK

SEQ ID NO: 295 - protein, 11 aa, synthetic construct
  REGION 1..11: note = synthetic construct
QASQSIYRYLS

SEQ ID NO: 296 - protein, 7 aa, synthetic construct
  REGION 1..7: note = synthetic construct
GASNLAS

SEQ ID NO: 297 - protein, 14 aa, synthetic construct
  REGION 1..14: note = synthetic construct
QSYYYSSSSGYGNV

SEQ ID NO: 298 - protein, 122 aa, synthetic construct
  REGION 1..122: note = synthetic construct
QEQLKESGGGLVQPGGSLKLSCKASGFDLSSYGVSWVRQAPGKGLEWIGYIDPVFGSTYYANWVNGRFTISSDNAQNTLYLQLNSLTAADTATYFCARGSGYVYYGYTYDLWGPGTLVTVSS

SEQ ID NO: 299 - protein, 10 aa, synthetic construct
  REGION 1..10: note = synthetic construct
GFDLSSYGVS

SEQ ID NO: 300 - protein, 17 aa, synthetic construct
  REGION 1..17: note = synthetic construct
YIDPVFGSTYYANWVNG

SEQ ID NO: 301 - protein, 13 aa, synthetic construct
  REGION 1..13: note = synthetic construct
GSGYVYYGYTYDL

SEQ ID NO: 302 - protein, 113 aa, synthetic construct
  REGION 1..113: note = synthetic construct
DIVMTQSPSSLAMSVGQKVTMSCKSSQSLLSSYNQKNYLAWYQRKPGQSPKLLVYFASQRATGVPDRFIGSGSGSDFTLTISSVQAEDLADYFCQQHTRLPITFGAGTKLELK

SEQ ID NO: 303 - protein, 17 aa, synthetic construct
  REGION 1..17: note = synthetic construct
KSSQSLLSSYNQKNYLA

SEQ ID NO: 304 - protein, 7 aa, synthetic construct
  REGION 1..7: note = synthetic construct
FASQRAT

SEQ ID NO: 305 - protein, 9 aa, synthetic construct
  REGION 1..9: note = synthetic construct
QQHTRLPIT

SEQ ID NO: 306 - protein, 119 aa, synthetic construct
  REGION 1..119: note = synthetic construct
EVQLQQSGPVLVKPGASVKMSCKASGYPFNDYFLGWVKQSHGKSLEWIGSINPDNDYTMSNQKFKGKATLTVDKSSSTAYMELNSLTSEDSAVYYCARYDYDWAWFAHWGQGTLVTVSA

SEQ ID NO: 307 - protein, 10 aa, synthetic construct
  REGION 1..10: note = synthetic construct
GYPFNDYFLG

SEQ ID NO: 308 - protein, 17 aa, synthetic construct
  REGION 1..17: note = synthetic construct
SINPDNDYTMSNQKFKG

SEQ ID NO: 309 - protein, 10 aa, synthetic construct
  REGION 1..10: note = synthetic construct
YDYDWAWFAH

SEQ ID NO: 310 - protein, 216 aa, synthetic construct
  REGION 1..216: note = synthetic construct
DIVMTQTPASVEAAVGGTVTIKCQASQSIYRYLSWYQQKPGQRPKLLIYGASNLASGVSSRFKGSGSGTEFTLTISDLECADAATYYCQSYYYSSSSGYGNVFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 311 - protein, 445 aa, synthetic construct
  REGION 1..445: note = synthetic construct
QEQLKESGGGLVQPGGSLKLSCKASGFDLSSYGVSWVRQAPGKGLEWIGYIDPVFGSTYYANWVNGRFTISSDNAQNTLYLQLNSLTAADTATYFCARGSGYVYYGYTYDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 312 - protein, 220 aa, synthetic construct
  REGION 1..220: note = synthetic construct
DIVMTQSPSSLAMSVGQKVTMSCKSSQSLLSSYNQKNYLAWYQRKPGQSPKLLVYFASQRATGVPDRFIGSGSGSDFTLTISSVQAEDLADYFCQQHTRLPITFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 313 - protein, 443 aa, synthetic construct
  REGION 1..443: note = synthetic construct
EVQLQQSGPVLVKPGASVKMSCKASGYPFNDYFLGWVKQSHGKSLEWIGSINPDNDYTMSNQKFKGKATLTVDKSSSTAYMELNSLTSEDSAVYYCARYDYDWAWFAHWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK